(19) United States
(12) Patent Application Publication
(10) Pub. No.: US 2012/0195910 A1
(43) Pub. Date: Aug. 2, 2012

(54) ANTI-PCSK9 ANTIBODIES AND METHODS OF USE

(75) Inventors: YAN WU, SAN FRANCISCO, CA (US); CECILIA CHIU, SAN CARLOS, CA (US); DANIEL KIRCHHOFER, LOS ALTOS, CA (US); ANDREW PETERSON, SAN FRANCISCO, CA (US); GANESH KOLUMAM, FOSTER CITY, CA (US); MONICA KONG BELTRAN, Foster City, CA (US); PAUL MORAN, Oakland, CA (US); WEI LI, PALO ALTO, CA (US)

(73) Assignee: GENENTECH, INC., SOUTH SAN FRANCISCO, CA (US)

(21) Appl. No.: 13/333,315

(22) Filed: Dec. 21, 2011

Related U.S. Application Data
(60) Provisional application No. 61/477,788, filed on Apr. 21, 2011.
(60) Provisional application No. 61/426,343, filed on Dec. 22, 2010.

(51) Int. Cl. A61K 39/395; C12N 5/10; C12N 1/21; A61P 7/00; C12N 1/19; C12N 15/63; C07K 16/40; C12N 15/13; G01N 33/573; C12N 1/15

(52) U.S. Cl. 424/158.1; 435/7.4; 435/325; 435/348; 435/350; 435/352; 435/353; 435/354; 435/358; 435/364; 435/366; 435/369; 435/370; 435/419; 435/252.33; 435/254.11; 435/254.2; 435/320.1; 530/387.3; 530/388.26; 530/389.1; 536/23.53

(57) ABSTRACT

The invention provides anti-PCSK9 antibodies and methods of using the same.

### RELATED APPLICATIONS

This application claims the benefit of U.S. Provisional Application Ser. No. 61/477,788, filed Apr. 21, 2011 and 61/426,343, filed Dec. 22, 2010. All the teachings of the above-referenced applications are incorporated herein by reference.

### SEQUENCE LISTING

The instant application contains a Sequence Listing which has been submitted in ASCII format via EFS-Web and is hereby incorporated by reference in its entirety. Said ASCII copy, created on Dec. 12, 2011, is named P4539RUS.txt and is 27,655 bytes in size.

### FIELD OF THE INVENTION

The present invention relates to anti-PCSK9 antibodies and methods of using the same.

### BACKGROUND OF THE INVENTION

Proprotein convertase subtilisin/kexin type 9 (PCSK9) is a member of the mammalian subtilisin family of proprotein convertases. PCSK9 plays a critical role in cholesterol metabolism by controlling the levels of low density lipoprotein (LDL) particles that circulate in the bloodstream. Elevated levels of PCSK9 have been shown to reduce LDL-receptor levels in the liver, resulting in high levels of LDL-cholesterol in the plasma and increased susceptibility to coronary artery disease. (Peterson et al., J Lipid Res. 49(7):1595-9 (2008)). Therefore, it would be highly advantageous to produce a therapeutic-based antagonist of PCSK9 that inhibits or antagonizes the activity of PCSK9 and the corresponding role PCSK9 plays in various therapeutic conditions.

### SUMMARY OF THE INVENTION

The invention is in part based on a variety of antibodies to PCSK9. PCSK9 presents as an important and advantageous therapeutic target, and the invention provides antibodies as therapeutic and diagnostic agents for use in targeting pathological conditions associated with expression and/or activity of PCSK9. Accordingly, the invention provides methods, compositions, kits and articles of manufacture related to PCSK9.

In certain embodiments, an antibody or an antibody fragment that binds to PCSK9 or a fragment thereof is provided, wherein the antibody comprises a variable domain comprising at least one, two, three, four, five or six hypervariable region (HVR) sequences selected from the group consisting of:

(i) HVR-H1 comprising GFTFX₁X₂X₃X₄IH (SEQ ID NO: 28), wherein X₁ is S or T; X₂ is G, R or S; X₃ is H, T or Y; X₄ is A or T;

(ii) HVR-H2 comprising RISPANGNTNYADSVKG (SEQ ID NO:4);

(iii) HVR-H3 comprising WIGSRELYIMDY (SEQ ID NO:5);

(iv) HVR-L1 comprising RASQDVSX₁AVA (SEQ ID NO:29), wherein X₁ is S or T;

(v) HVR-L2 comprising SASX₁LYS (SEQ ID NO:30), wherein X₁ is F or S; and

(vi) HVR-L3 comprising QQSYX₁X₂X₃X₄T (SEQ ID NO:31), wherein X₁ is P, R or T; X₂ is A, I, S or T; X₃ is L, P or Q; X₄ is A, H, P or S.

In certain embodiments, an antibody or an antibody fragment that binds to PCSK9 or a fragment thereof is provided, wherein the antibody comprises a variable domain comprising the following six HVR sequences:

(i) HVR-H1 comprising GFTFX₁X₂X₃X₄₁H (SEQ ID NO:28), wherein X₁ is S or T; X₂ is G, R or S; X₃ is H, T or Y; X₄ is A or T;

(ii) HVR-H2 comprising RISPANGNTNYADSVKG (SEQ ID NO:4);

(iii) HVR-H3 comprising WIGSRELYIMDY (SEQ ID NO:5);

(iv) HVR-L1 comprising RASQDVSX₁AVA (SEQ ID NO:29), wherein X₁ is S or T;

(v) HVR-L2 comprising SASX₁LYS (SEQ ID NO:30), wherein X₁ is F or S; and

(vi) HVR-L3 comprising QQSYX₁X₂X₃X₄T (SEQ ID NO:31), wherein X₁ is P, R or T; X₂ is A, I, S or T; X₃ is L, P or Q; X₄ is A, H, P or S.

In certain embodiments, an antibody or an antibody fragment that binds to PCSK9 or a fragment thereof is provided, wherein the antibody comprises (a) HVR-H1 comprising the amino acid sequence of SEQ ID NO:1, SEQ ID NO:2 or SEQ ID NO:3, (b) HVR-H2 comprising the amino acid sequence of SEQ ID NO:4, and (c) HVR-H3 comprising the amino acid sequence of SEQ ID NO:5. In certain embodiments, the antibody further comprises (a) HVR-L1 comprising the amino acid sequence of SEQ ID NO:6 or SEQ ID NO:7; (b) HVR-L2 comprising the amino acid sequence of SEQ ID NO:8 or SEQ ID NO:26; and (c) HVR-L3 comprising the amino acid sequence of SEQ ID NO:9, SEQ ID NO:10, SEQ ID NO:11, SEQ ID NO:12, SEQ ID NO:13 or SEQ ID NO:14.

In certain embodiments, an antibody or an antibody fragment that binds to PCSK9 or a fragment thereof is provided, wherein the antibody comprises (a) HVR-L1 comprising the amino acid sequence of SEQ ID NO:6 or SEQ ID NO:7; (b) HVR-L2 comprising the amino acid sequence of SEQ ID NO:8 or SEQ ID NO:26; and (c) HVR-L3 comprising the amino acid sequence of SEQ ID NO: 9, SEQ ID NO:10, SEQ ID NO:11, SEQ ID NO:12, SEQ ID NO:13 or SEQ ID NO:14. In certain embodiments, the antibody further comprises (a) HVR-H1 comprising the amino acid sequence of SEQ ID NO:1, SEQ ID NO:2 or SEQ ID NO:3, (b) HVR-H2 comprising the amino acid sequence of SEQ ID NO:4, and (c) HVR-H3 comprising the amino acid sequence of SEQ ID NO:5.

In one embodiment, an antibody or an antibody fragment that binds to PCSK9 or a fragment thereof is provided, wherein the antibody comprises:

(1) an HVR-H1 comprising the amino acid sequence of SEQ ID NO:1;

(2) an HVR-H2 comprising the amino acid sequence of SEQ ID NO:4;

(3) an HVR-H3 comprising the amino acid sequence of SEQ ID NO:5;

(4) an HVR-L1 comprising the amino acid sequence of SEQ ID NO:6;

(5) an HVR-L2 comprising the amino acid sequence of SEQ ID NO:26; and

(6) an HVR-L3 comprising the amino acid sequence of SEQ ID NO:9.

In one embodiment, an antibody or an antibody fragment that binds to PCSK9 or a fragment thereof is provided, wherein the antibody comprises:

(1) an HVR-H1 comprising the amino acid sequence of SEQ ID NO:1;

(2) an HVR-H2 comprising the amino acid sequence of SEQ ID NO:4;

(3) an HVR-H3 comprising the amino acid sequence of SEQ ID NO:5;

(4) an HVR-L1 comprising the amino acid sequence of SEQ ID NO:7;

(5) an HVR-L2 comprising the amino acid sequence of SEQ ID NO:8; and

(6) an HVR-L3 comprising the amino acid sequence of SEQ ID NO:10.

In another embodiment, an antibody or an antibody fragment that binds to PCSK9 or a fragment thereof is provided, wherein the antibody comprises:

(1) an HVR-H1 comprising the amino acid sequence of SEQ ID NO:1;

(2) an HVR-H2 comprising the amino acid sequence of SEQ ID NO:4;

(3) an HVR-H3 comprising the amino acid sequence of SEQ ID NO:5;

(4) an HVR-L1 comprising the amino acid sequence of SEQ ID NO:7;

(5) an HVR-L2 comprising the amino acid sequence of SEQ ID NO:8; and

(6) an HVR-L3 comprising the amino acid sequence of SEQ ID NO:11.

In another embodiment, an antibody or an antibody fragment that binds to PCSK9 or a fragment thereof is provided, wherein the antibody comprises:

(1) an HVR-H1 comprising the amino acid sequence of SEQ ID NO:2;

(2) an HVR-H2 comprising the amino acid sequence of SEQ ID NO:4;

(3) an HVR-H3 comprising the amino acid sequence of SEQ ID NO:5;

(4) an HVR-L1 comprising the amino acid sequence of SEQ ID NO:7;

(5) an HVR-L2 comprising the amino acid sequence of SEQ ID NO:8; and

(6) an HVR-L3 comprising the amino acid sequence of SEQ ID NO:12.

In another embodiment, an antibody or an antibody fragment that binds to PCSK9 or a fragment thereof is provided, wherein the antibody comprises:

(1) an HVR-H1 comprising the amino acid sequence of SEQ ID NO:3;

(2) an HVR-H2 comprising the amino acid sequence of SEQ ID NO:4;

(3) an HVR-H3 comprising the amino acid sequence of SEQ ID NO:5;

(4) an HVR-L1 comprising the amino acid sequence of SEQ ID NO:7;

(5) an HVR-L2 comprising the amino acid sequence of SEQ ID NO:8; and

(6) an HVR-L3 comprising the amino acid sequence of SEQ ID NO:13.

In another embodiment, an antibody or an antibody fragment that binds to PCSK9 or a fragment thereof is provided, wherein the antibody comprises:

(1) an HVR-H1 comprising the amino acid sequence of SEQ ID NO:1;

(2) an HVR-H2 comprising the amino acid sequence of SEQ ID NO:4;

(3) an HVR-H3 comprising the amino acid sequence of SEQ ID NO:5;

(4) an HVR-L1 comprising the amino acid sequence of SEQ ID NO:7;

(5) an HVR-L2 comprising the amino acid sequence of SEQ ID NO:8; and

(6) an HVR-L3 comprising the amino acid sequence of SEQ ID NO:14.

In certain embodiments, an antibody or an antibody fragment that binds to PCSK9 or a fragment thereof is provided, wherein the antibody comprises (a) a VH sequence having at least 95% sequence identity to the amino acid sequence of SEQ ID NO:15, SEQ ID NO:16, SEQ ID NO:17, or SEQ ID NO:27; or (b) a VL sequence having at least 95% sequence identity to the amino acid sequence of SEQ ID NO: 18, SEQ ID NO:19, SEQ ID NO:20, SEQ ID NO:21, SEQ ID NO:22 or SEQ ID NO:23.

In certain embodiments, an antibody or an antibody fragment that binds to PCSK9 or a fragment thereof is provided, wherein the antibody comprises a VH sequence of SEQ ID NO:15, SEQ ID NO:16, SEQ ID NO:17, or SEQ ID NO:27. In certain embodiments, the antibody further comprises a VL sequence of SEQ ID NO: 18, SEQ ID NO:19, SEQ ID NO:20, SEQ ID NO:21, SEQ ID NO:22 or SEQ ID NO:23.

In one embodiment, an antibody or an antibody fragment that binds to PCSK9 or a fragment thereof is provided, wherein the antibody comprises a VH sequence of SEQ ID NO:15 and a VL sequence of SEQ ID NO:18. In another embodiment, an antibody or an antibody fragment that binds to PCSK9 or a fragment thereof is provided, wherein the antibody comprises a VH sequence of SEQ ID NO:15 and a VL sequence of SEQ ID NO:19. In another embodiment, an antibody or an antibody fragment that binds to PCSK9 or a fragment thereof is provided, wherein the antibody comprises a VH sequence of SEQ ID NO:27 and a VL sequence of SEQ ID NO:20. In another embodiment, an antibody or an antibody fragment that binds to PCSK9 or a fragment thereof is provided, wherein the antibody comprises a VH sequence of SEQ ID NO:16 and a VL sequence of SEQ ID NO:21. In another embodiment, an antibody or an antibody fragment that binds to PCSK9 or a fragment thereof is provided, wherein the antibody comprises a VH sequence of SEQ ID NO:17 and a VL sequence of SEQ ID NO:22. In another embodiment, an antibody or an antibody fragment that binds to PCSK9 or a fragment thereof is provided, wherein the antibody comprises a VH sequence of SEQ ID NO:27 and a VL sequence of SEQ ID NO:23.

In certain embodiments, an antibody or an antibody fragment that binds to PCSK9 or a fragment thereof is provided, wherein the antibody binds to an epitope within a fragment of PCSK9. In certain embodiments, an antibody or an antibody fragment that binds to PCSK9 or a fragment thereof is provided, wherein the antibody binds to an epitope within a fragment of PCSK9 comprising amino acids 376 to 379 of human PCSK9 amino acid sequence of SEQ ID NO:24. In certain embodiments, the functional and/or structural epitope of an antibody according to this invention includes residue D238 of human PCSK9. In certain embodiments, the functional and/or structural epitope of an antibody according to this invention includes residue A239 of human PCSK9. In certain embodiments, the functional and/or structural epitope of an antibody according to this invention includes residues D238 and A239 of human PCSK9. In certain embodiments, the functional and/or structural epitope of an antibody according to this invention includes residue E366 of human PCSK9. In certain embodiments, the functional and/or structural epitope of an antibody according to this invention includes residue D367 of human PCSK9. In certain embodiments, the functional and/or structural epitope of an antibody according to this invention includes residues E366 and D367 of human PCSK9. In certain embodiments, the functional and/or structural epitope of an antibody according to this invention includes residue H391 of human PCSK9. In certain embodiments, the functional and/or structural epitope of an antibody according to this invention includes residues E366, D367 and H391 of human PCSK9. According to another embodiment, the functional and/or structural epitope of an antibody according to this invention includes residues A239 and H391 of human PCSK9. In certain embodiments, the functional and/or structural epitope of includes one or more of residues A239, A341, E366, D367 and H391 of human PCSK9. In certain embodiments, the functional and/or structural epitope of includes one or more of residues near A239, A341, E366, D367 and H391 of human PCSK9. In certain embodiments, the functional and/or structural epitope of an antibody according to this invention comprises (i) at least one residue selected from the group consisting of R194 and E195, (ii) at least one residue selected from the group consisting of D238 and A239, (iii) at least one residue selected from the group consisting of A341 and Q342, and (iv) at least one residue selected from the group consisting of E366, D367, 1369, S376, T377, C378, F379, S381 and H391, of human PCSK9. In certain embodiments, the functional and/or structural epitope comprises one, two, three, four, five, six, seven, eight, nine, ten, eleven, twelve, thirteen, fourteen or all of the following residues: R194, E195, D238, A239, A341, Q342, E366, D367, 1369, S376, T377, C378, F379, S381 and H391 of human PCSK9.

In certain embodiments, the anti-PCSK9 antibody is a monoclonal antibody. In certain embodiments, the anti-PCSK9 antibody is humanized. In certain embodiments, the anti-PCSK9 antibody is a human antibody. In certain embodiments, at least a portion of the framework sequence of the anti-PCSK9 antibody is a human consensus framework sequence. In one embodiment, the antibody is an antibody fragment selected from a Fab, Fab′-SH, Fv, scFv, or (Fab)₂ fragment.

In one aspect, a nucleic acid encoding any of the above anti-PCSK9 antibodies is provided. In one embodiment, a vector comprising the nucleic is provided. In one embodiment, the vector is an expression vector. In one embodiment, a host cell comprising the vector is provided. In one embodiment, the host cell is eukaryotic. In another embodiment, the host cell is mammalian. In yet another embodiment, the host cell is prokaryotic. In one embodiment, a method of making an anti-PCSK9 antibody is provided, wherein the method comprises culturing the host cell under conditions suitable for expression of the nucleic acid encoding the antibody, and isolating the antibody. In certain embodiment, the method further comprises recovering the anti-PCSK9 antibody from the host cell. In certain embodiments, a composition comprising any of the anti-PCSK9 antibodies described herein is provided. In one embodiment, the composition further comprises a pharmaceutically acceptable carrier.

In one aspect, the invention concerns methods of inhibiting binding of PCSK9 to LDL-receptor (LDLR) in a subject, said method comprising administering to the subject an effective amount of any of the anti-PCSK9 antibodies described herein. In another aspect, the invention concerns methods of reducing a level of cholesterol in a subject, said method comprising administering to the subject an effective amount of any of the anti-PCSK9 antibodies described herein. In one embodiment, the cholesterol is LDL-cholesterol. In another aspect, the invention concerns methods of reducing a level of LDL-cholesterol in a subject, said method comprising administering to the subject an effective amount of any of the anti-PCSK9 antibodies described herein. In certain embodiments, the invention concerns methods of lowering serum LDL-cholesterol level in a subject, said method comprising administering to the subject an effective amount of any one of the anti-PCSK9 antibodies described herein. In another aspect, the invention concerns methods of treating a condition associated with elevated level of LDL-cholesterol in a subject, said method comprising administering to the subject an effective amount of any one of the anti-PCSK9 antibodies described herein.

In one aspect, the invention concerns methods of treating a cholesterol related disorder. An exemplary and non-limiting list of cholesterol related disorders contemplated is provided herein under “Compositions and Methods.” In certain embodiments, the cholesterol related disorder is hypercholesterolemia. In certain embodiments, the invention concerns methods of treating hypercholesterolemia comprising administering to the subject an effective amount of any one of the anti-PCSK9 antibodies described herein. In certain embodiments, the invention concerns methods of preventing and/or treating atherosclerosis and/or cardiovascular diseases. In certain embodiments, the invention concerns methods of reducing the risk of recurrent cardiovascular events in an individual comprising administering to the individual an amount effective of any one of the anti-PCSK9 antibodies described herein.

In one aspect, the invention concerns methods for treating any disease or condition which can be improved, ameliorated, inhibited or prevented by removal, inhibition or reduction of PCSK9 activity. In certain embodiments, diseases or disorders that are either treatable or preventable through the use of statins can also be treated using any one of the anti-PCSK9 antibodies described herein. In certain embodiments, disorders or disease that can benefit from the prevention of cholesterol synthesis or increased LDLR expression can also be treated using any one of the anti-PCSK9 antibodies described herein.

In certain embodiments, the methods described herein further comprise administering to the subject an effective amount of a second medicament, wherein the anti-PCSK9 antibody is the first medicament. In one embodiment, the second medicament elevates the level of LDLR protein. In another embodiment, the second medicament reduces the level of LDL-cholesterol. In another embodiment, the second medicament comprises a statin. In another embodiment, the statin is selected from the group consisting of atorvastatin, fluvastatin, lovastatin, mevastatin, pitavastatin, pravastatin, rosuvastatin, simvastatin, and any combination thereof. In another embodiment, the second medicament elevates the level of HDL-cholesterol. In certain embodiments, the subject or the individual is human.

In one aspect, the invention concerns a method of detecting PCSK9 protein in a sample suspected of containing the PCSK9 protein, the method comprising (a) contacting the sample with the anti-PCSK9 antibody described herein; and (b) detecting formation of a complex between the anti-PCSK9 antibody and the PCSK9 protein. In one embodiment, the anti-PCSK9 antibody is detectably labeled.

Any embodiment described herein or any combination thereof applies to any and all anti-PCSK9 antibodies, methods and uses of the invention described herein.

### BRIEF DESCRIPTION OF THE FIGURES

FIG. 1 shows heavy chain HVR sequences, H1 (SEQ ID NOS 1, 1, 1-3 and 1, respectively, in order of appearance), H2 (all disclosed as SEQ ID NO: 4), and H3 (all disclosed as SEQ ID NO: 5), and light chain HVR sequences, L1 (SEQ ID NOS: 6, 7, 7, 7, 7 and 7, respectively, in order of appearance), L2 (SEQ ID NOS: 26, 8, 8, 8, 8 and 8, respectively, in order of appearance) and L3 (SEQ ID NOS: 9-14, respectively, in order of appearance), of anti-PCSK9 antibodies.

FIG. 2A-B show the amino acid sequences of (A) the heavy chain variable domains (SEQ ID NOS: 15, 15, 27, 16-17 and 27, respectively, in order of appearance) and (B) light chain variable domains (SEQ ID NOS: 18-23, respectively, in order of appearance) of anti-PCSK9 antibodies. Positions are numbered according to Kabat and hypervariable regions are boxed.

FIG. 3A-D show dissociation constants of the anti-PCSK9 antibodies (IgG) against (A) human PCSK9, (B) murine PCSK9, (C) cyno PCSK9 and rat PCSK9, and (D) rhesus PCSK9.

FIG. 4. Anti-PCSK9 antibodies inhibit binding of PCSK9 to LDLR in a competition binding ELISA. Blank (no antibody; open square) and control antibody (open circle) are shown in dashed lines. Anti-PCSK9 antibodies are shown in solid lines. IC₅₀ values of anti-PCSK9 antibodies are shown in the table.

FIG. 5. Different concentrations of anti-PCSK9 antibodies were incubated with 15 μg/ml PCSK9 and added to HepG2 cells for 4 hours. Cells were processed for FACS analysis of surface LDLR. The data indicate that the anti-PCSK9 antibodies effectively prevented LDLR downregulation. The positive control is cells not treated with PCSK9.

FIG. 6. Western blot with anti-LDLR antibody showing that 30 μg of PCSK9 for 1 hr significantly downregulated LDLR levels in mouse liver.

FIG. 7. Western blot with anti-LDLR antibody showing that all five anti-PCSK9 antibodies prevented LDLR downregulation in mouse liver. The bottom immunoblot is a pool of 4 livers (10 μg of protein from each liver) per treatment group.

FIG. 8 shows anti-PCSK9 antibody concentrations in sera of C57JBL/6 mice after single I.V. injection. Shown are the average concentrations of the dosing groups 0.5 mg/kg; 5 mg/kg; and 20 mg/kg (n=3).

FIG. 9 shows comparison of anti-PCSK9 antibody concentrations in sera of C57JBL/6 WT and PCSK9^{-/-} mice after single I.V. injection of 5 mg/kg anti-PCSK9 antibody. The average concentrations of each dosing group are shown (n=3).

FIG. 10 shows anti-PCSK9 antibody concentrations in sera of individual cynomolgus monkey after single I.V. injection. Three dosing groups are included: 5 mg/kg; 20 mg/kg; and 60 mg/kg.

FIG. 11 shows anti-PCSK9 antibody concentrations in sera of cynomolgus monkeys after single I.V. injection. Shown are the average concentrations of the dosing groups 5 mg/kg, 20 mg/kg, and 60 mg/kg (n=3).

FIG. 12 shows total cholesterol level in the sera of mice treated with a single dose (10 mg/kg body weight) of either control (Crtl) or anti-PCSK9 antibody. Cholesterol levels were measured at different days as indicated in the figure.

FIG. 13 shows total cholesterol level in the sera from the mice treated with single dose (10 mg/kg body weight) of either control or anti-PCSK9 antibody.

### DETAILED DESCRIPTION OF EMBODIMENTS OF THE INVENTION

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

### I. DEFINITIONS

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application. All references cited herein, including patent applications and publications, are incorporated by reference in their entirety.

For purposes of interpreting this specification, the following definitions will apply and whenever appropriate, terms used in the singular will also include the plural and vice versa. It is to be understood that the terminology used herein is for the purpose of describing particular embodiments only, and is not intended to be limiting. In the event that any definition set forth below conflicts with any document incorporated herein by reference, the definition set forth below shall control.

Throughout the present specification and claims, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG₁ EU antibody.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-PCSK9 antibody”, “anti-PCSK9”, “PCSK9 antibody” or “an antibody that binds to PCSK9” refers to an antibody that is capable of binding PCSK9 with sufficient affinity such that the antibody is useful as a diagnostic and/or therapeutic agent in targeting PCSK9. In one embodiment, the extent of binding of an anti-PCSK9 antibody to an unrelated, non-PCSK9 protein is less than about 10% of the binding of the antibody to PCSK9 as measured, e.g., by a radioimmunoassay (RIA). In certain embodiments, an antibody that binds to PCSK9 has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M). In certain embodiments, an anti-PCSK9 antibody binds to an epitope of PCSK9 that is conserved among PCSK9 from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein. In certain embodiments, the epitope is determined based on the crystal structure of the anti-PCSK9 antibody Fab fragment bound to PCSK9.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively. The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

The term “diabodies” refers to antibody fragments with two antigen-binding sites, which fragments comprise a heavy-chain variable domain (VH) connected to a light-chain variable domain (VL) in the same polypeptide chain (V_{H}-V_{L}). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The “Fab” fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fc region” herein is used to define a C-terminal region of an immunoglobulin heavy chain that contains at least a portion of the constant region. The term includes native sequence Fc regions and variant Fc regions. In certain embodiments, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVRand FR sequences generally appear in the following sequence in VH (or VL): FR1-H1(L1)-FR2-H2(L2)-FR3-H3 (L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypercholesterolemia,” as used herein, refers to a condition in which cholesterol levels are elevated above a desired level. In certain embodiments, the LDL-cholesterol level is elevated above the desired level. In certain embodiments, the serum LDL-cholesterol levels are elevated above the desired level.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH(H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-PCSK9 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody,” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “proprotein convertase subtilisin kexin type 9,” “PCSK9,” or “NARC-1,” as used herein, refers to any native PCSK9 from any vertebrate source, including mammals such as primates (e.g. humans) and rodents (e.g., mice and rats), unless otherwise indicated. The term encompasses “full-length,” unprocessed PCSK9 as well as any form of PCSK9 that results from processing in the cell or any fragment thereof. The term also encompasses naturally occurring variants of PCSK9, e.g., splice variants or allelic variants.

The term “PCSK9 activity” or “biological activity” of PCSK9, as used herein, includes any biological effect of PCSK9. In certain embodiments, PCSK9 activity includes the ability of PCSK9 to interact or bind to a substrate or receptor. In certain embodiments, the biological activity of PCSK9 is the ability of PCSK9 to bind to a LDL-receptor (LDLR). In certain embodiments, PCSK9 binds to and catalyzes a reaction involving LDLR. In certain embodiments, PCSK9 activity includes the ability of PCSK9 to decrease or reduce the availability of LDLR. In certain embodiments, the biological activity of PCSK9 includes the ability of PCSK9 to increase the amount of LDL in a subject. In certain embodiments, the biological activity of PCSK9 includes the ability of PCSK9 to decrease the amount of LDLR that is available to bind to LDL in a subject. In certain embodiments, the biological activity of PCSK9 includes the ability of PCSK9 to decrease the amount of LDLR that is available to bind to LDL. In certain embodiments, biological activity of PCSK9 includes any biological activity resulting from PCSK9 signaling.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthiin, in The Pharmacology of Monoclonal Antibodies, vol. 113, Rosenburg and Moore eds., (Springer-Verlag, New York, 1994), pp. 269-315.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. COMPOSITIONS AND METHODS

In one aspect, the invention is based, in part, on experimental results obtained with anti-PCSK9 antibodies. Results obtained indicate that blocking biological activity of PCSK9 with anti-PCSK9 antibodies leads to a prevention of reduction in LDLR. In addition, the results demonstrate that administration of anti-PCSK9 antibody reduces total LDL-cholesterol level in a subject. Accordingly, PCSK9 antibodies of the invention, as described herein, provide important therapeutic and diagnostic agents for use in targeting pathological conditions associated with PCSK9, e.g., cholesterol related disorders.

In certain embodiments, a “cholesterol related disorder” includes any one or more of the following: hypercholesterolemia, heart disease, metabolic syndrome, diabetes, coronary heart disease, stroke, cardiovascular diseases, Alzheimers disease and generally dyslipidemias, which can be manifested, for example, by an elevated total serum cholesterol, elevated LDL, elevated triglycerides, elevated VLDL, and/or low HDL. Some non-limiting examples of primary and secondary dyslipidemias that can be treated using an anti-PCSK9 antibody, either alone, or in combination with one or more other agents include the metabolic syndrome, diabetes mellitus, familial combined hyperlipidemia, familial hypertriglyceridemia, familial hypercholesterolemias, including heterozygous hypercholesterolemia, homozygous hypercholesterolemia, familial defective apoplipoprotein B-100; polygenic hypercholesterolemia; remnant removal disease, hepatic lipase deficiency; dyslipidemia secondary to any of the following: dietary indiscretion, hypothyroidism, drugs including estrogen and progestin therapy, beta-blockers, and thiazide diuretics; nephrotic syndrome, chronic renal failure, Cushing's syndrome, primary biliary cirrhosis, glycogen storage diseases, hepatoma, cholestasis, acromegaly, insulinoma, isolated growth hormone deficiency, and alcohol-induced hypertriglyceridemia. Anti-PCSK9 antibodies described herein can also be useful in preventing or treating atherosclerotic diseases, such as, for example, coronary heart disease, coronary artery disease, peripheral arterial disease, stroke (ischaemic and hemorrhagic), angina pectoris, or cerebrovascular disease and acute coronary syndrome, myocardial infarction. In certain embodiments, the anti-PCSK9 antibodies described herein are useful in reducing the risk of: nonfatal heart attacks, fatal and non-fatal strokes, certain types of heart surgery, hospitalization for heart failure, chest pain in patients with heart disease, and/or cardiovascular events because of established heart disease such as prior heart attack, prior heart surgery, and/or chest pain with evidence of clogged arteries. In certain embodiments, the anti-PCSK9 antibodies and methods described herein can be used to reduce the risk of recurrent cardiovascular events.

A. Exemplary Anti-PC SK9 Antibodies

In one aspect, the invention provides isolated antibodies that bind to PCSK9. In certain embodiments, an anti-PCSK9 antibody modulates PCSK9 activity.

In one aspect, the invention provides an anti-PCSK9 antibody comprising at least one, two, three, four, five, or six HVRs selected from (a) HVR-H1 comprising the amino acid sequence of SEQ ID NO:1, SEQ ID NO:2 or SEQ ID NO:3; (b) HVR-H2 comprising the amino acid sequence of SEQ ID NO:4; (c) HVR-H3 comprising the amino acid sequence of SEQ ID NO:5; (d) HVR-L1 comprising the amino acid sequence of SEQ ID NO:6 or SEQ ID NO:7; (e) HVR-L2 comprising the amino acid sequence of SEQ ID NO:8 or SEQ ID NO:26; and (f) HVR-L3 comprising the amino acid sequence of SEQ ID NO:9, SEQ ID NO:10, SEQ ID NO:11, SEQ ID NO:12, SEQ ID NO:13 or SEQ ID NO:14.

In one aspect, the invention provides an anti-PCSK9 antibody comprising six HVRs comprising (a) HVR-H1 comprising the amino acid sequence of SEQ ID NO:1, SEQ ID NO:2 or SEQ ID NO:3; (b) HVR-H2 comprising the amino acid sequence of SEQ ID NO:4; (c) HVR-H3 comprising the amino acid sequence of SEQ ID NO:5; (d) HVR-L1 comprising the amino acid sequence of SEQ ID NO:6 or SEQ ID NO:7; (e) HVR-L2 comprising the amino acid sequence of SEQ ID NO:8 or SEQ ID NO:26; and (f) HVR-L3 comprising the amino acid sequence of SEQ ID NO:9, SEQ ID NO:10, SEQ ID NO:11, SEQ ID NO:12, SEQ ID NO:13 or SEQ ID NO:14.

In one aspect, the invention provides an antibody comprising at least one, at least two, or all three VH HVR sequences selected from (a) HVR-H1 comprising the amino acid sequence of SEQ ID NO:1, SEQ ID NO:2 or SEQ ID NO:3; (b) HVR-H2 comprising the amino acid sequence of SEQ ID NO:4; and (c) HVR-H3 comprising the amino acid sequence of SEQ ID NO:5. In one embodiment, the antibody comprises HVR-H3 comprising the amino acid sequence of SEQ ID NO:5. In another embodiment, the antibody comprises HVR-H3 comprising the amino acid sequence of SEQ ID NO:5 and HVR-L3 comprising the amino acid sequence of SEQ ID NO:9, SEQ ID NO:10, SEQ ID NO:11, SEQ ID NO:12, SEQ ID NO:13 or SEQ ID NO:14. In a further embodiment, the antibody comprises HVR-H3 comprising the amino acid sequence of SEQ ID NO:5, HVR-L3 comprising the amino acid sequence of SEQ ID NO:9, SEQ ID NO:10, SEQ ID NO:11, SEQ ID NO:12, SEQ ID NO:13 or SEQ ID NO:14, and HVR-H2 comprising the amino acid sequence of SEQ ID NO:4. In a further embodiment, the antibody comprises (a) HVR-H1 comprising the amino acid sequence of SEQ ID NO:1, SEQ ID NO:2 or SEQ ID NO:3; (b) HVR-H2 comprising the amino acid sequence of SEQ ID NO:4; and (c) HVR-H3 comprising the amino acid sequence of SEQ ID NO:5.

In another aspect, the invention provides an antibody comprising at least one, at least two, or all three VL HVR sequences selected from (a) HVR-L1 comprising the amino acid sequence of SEQ ID NO:6 or SEQ ID NO:7; (b) HVR-L2 comprising the amino acid sequence of SEQ ID NO:8 or SEQ ID NO:26; and (c) HVR-L3 comprising the amino acid sequence of SEQ ID NO:9, SEQ ID NO:10, SEQ ID NO:11, SEQ ID NO:12, SEQ ID NO:13 or SEQ ID NO:14. In one embodiment, the antibody comprises (a) HVR-L1 comprising the amino acid sequence of SEQ ID NO:6 or SEQ ID NO:7; (b) HVR-L2 comprising the amino acid sequence of SEQ ID NO:8 or SEQ ID NO:26; and (c) HVR-L3 comprising the amino acid sequence of SEQ ID NO:9, SEQ ID NO:10, SEQ ID NO:11, SEQ ID NO:12, SEQ ID NO:13 or SEQ ID NO:14.

In another aspect, an antibody of the invention comprises (a) a VH domain comprising at least one, at least two, or all three VH HVR sequences selected from (i) HVR-H1 comprising the amino acid sequence of SEQ ID NO:1, SEQ ID NO:2 or SEQ ID NO:3, (ii) HVR-H2 comprising the amino acid sequence of SEQ ID NO:4, and (iii) HVR-H3 comprising an amino acid sequence selected from SEQ ID NO:5; and (b) a VL domain comprising at least one, at least two, or all three VL HVR sequences selected from (i) HVR-L1 comprising the amino acid sequence of SEQ ID NO:6 or SEQ ID NO:7, (ii) HVR-L2 comprising the amino acid sequence of SEQ ID NO:8 or SEQ ID NO:26, and (c) HVR-L3 comprising the amino acid sequence of SEQ ID NO:9, SEQ ID NO:10, SEQ ID NO:11, SEQ ID NO:12, SEQ ID NO:13 or SEQ ID NO:14.

In another aspect, the invention provides an antibody comprising (a) HVR-H1 comprising the amino acid sequence of SEQ ID NO:1; (b) HVR-H2 comprising the amino acid sequence of SEQ ID NO:4; (c) HVR-H3 comprising the amino acid sequence of SEQ ID NO:5; (d) HVR-L1 comprising the amino acid sequence of SEQ ID NO:6; (e) HVR-L2 comprising the amino acid sequence of SEQ ID NO:26; and (f) HVR-L3 comprising an amino acid sequence of SEQ ID NO:9. In another aspect, the invention provides an antibody comprising (a) HVR-H1 comprising the amino acid sequence of SEQ ID NO:1; (b) HVR-H2 comprising the amino acid sequence of SEQ ID NO:4; (c) HVR-H3 comprising the amino acid sequence of SEQ ID NO:5; (d) HVR-L1 comprising the amino acid sequence of SEQ ID NO:7; (e) HVR-L2 comprising the amino acid sequence of SEQ ID NO:8; and (f) HVR-L3 comprising an amino acid sequence of SEQ ID NO:10. In another aspect, the invention provides an antibody comprising (a) HVR-H1 comprising the amino acid sequence of SEQ ID NO:1; (b) HVR-H2 comprising the amino acid sequence of SEQ ID NO:4; (c) HVR-H3 comprising the amino acid sequence of SEQ ID NO:5; (d) HVR-L1 comprising the amino acid sequence of SEQ ID NO:7; (e) HVR-L2 comprising the amino acid sequence of SEQ ID NO:8; and (f) HVR-L3 comprising an amino acid sequence of SEQ ID NO:11. In another aspect, the invention provides an antibody comprising (a) HVR-H1 comprising the amino acid sequence of SEQ ID NO:2; (b) HVR-H2 comprising the amino acid sequence of SEQ ID NO:4; (c) HVR-H3 comprising the amino acid sequence of SEQ ID NO:5; (d) HVR-L1 comprising the amino acid sequence of SEQ ID NO:7; (e) HVR-L2 comprising the amino acid sequence of SEQ ID NO:8; and (f) HVR-L3 comprising an amino acid sequence of SEQ ID NO:12. In another aspect, the invention provides an antibody comprising (a) HVR-H1 comprising the amino acid sequence of SEQ ID NO:3; (b) HVR-H2 comprising the amino acid sequence of SEQ ID NO:4; (c) HVR-H3 comprising the amino acid sequence of SEQ ID NO:5; (d) HVR-L1 comprising the amino acid sequence of SEQ ID NO:7; (e) HVR-L2 comprising the amino acid sequence of SEQ ID NO:8; and (f) HVR-L3 comprising an amino acid sequence of SEQ ID NO:13. In another aspect, the invention provides an antibody comprising (a) HVR-H1 comprising the amino acid sequence of SEQ ID NO:1; (b) HVR-H2 comprising the amino acid sequence of SEQ ID NO:4; (c) HVR-H3 comprising the amino acid sequence of SEQ ID NO:5; (d) HVR-L1 comprising the amino acid sequence of SEQ ID NO:7; (e) HVR-L2 comprising the amino acid sequence of SEQ ID NO:8; and (f) HVR-L3 comprising an amino acid sequence of SEQ ID NO:14.

In certain embodiments, the anti-PCSK9 antibody is humanized. In one embodiment, an anti-PCSK9 antibody comprises HVRs as in any of the above embodiments, and further comprises an acceptor human framework, e.g., a human immunoglobulin framework or a human consensus framework.

In another aspect, an anti-PCSK9 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:15, SEQ ID NO:16, SEQ ID NO:17 or SEQ ID NO:27. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-PCSK9 antibody comprising that sequence retains the ability to bind to PCSK9. In certain embodiments, a total of 1 to 10 amino acids have been substituted, inserted and/or deleted in SEQ ID NO:15, SEQ ID NO:16, SEQ NO:17 or or SEQ ID NO:27. In certain embodiments, substitutions, insertions, or deletions occur in regions outside the HVRs (i.e., in the FRs). Optionally, the anti-PCSK9 antibody comprises the VH sequence in SEQ ID NO:15, SEQ ID NO:16, SEQ ID NO:17, or or SEQ ID NO:27, including post-translational modifications of that sequence. In a particular embodiment, the VH comprises one, two or three HVRs selected from: (a) HVR-H1 comprising the amino acid sequence of SEQ ID NO:1, SEQ ID NO:2 or SEQ ID NO:3, (b) HVR-H2 comprising the amino acid sequence of SEQ ID NO:4, and (c) HVR-H3 comprising the amino acid sequence of SEQ ID NO:5.

In another aspect, an anti-PCSK9 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:18, SEQ ID NO:19, SEQ ID NO:20, SEQ ID NO:21, SEQ ID NO:22 or SEQ ID NO:23. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-PCSK9 antibody comprising that sequence retains the ability to bind to PCSK9. In certain embodiments, a total of 1 to 10 amino acids have been substituted, inserted and/or deleted in SEQ ID NO:18, SEQ ID NO:19, SEQ ID NO:20, SEQ ID NO:21, SEQ ID NO:22 or SEQ ID NO:23. In certain embodiments, the substitutions, insertions, or deletions occur in regions outside the HVRs (i.e., in the FRs). Optionally, the anti-PCSK9 antibody comprises the VL sequence in SEQ ID NO:18, SEQ ID NO:19, SEQ ID NO:20, SEQ ID NO:21, SEQ ID NO:22 or SEQ ID NO:23, including post-translational modifications of that sequence. In a particular embodiment, the VL comprises one, two or three HVRs selected from (a) HVR-L1 comprising the amino acid sequence of SEQ ID NO:6 or SEQ ID NO:7; (b) HVR-L2 comprising the amino acid sequence of SEQ ID NO:8 or SEQ ID NO:26; and (c) HVR-L3 comprising the amino acid sequence of SEQ ID NO:9, SEQ ID NO:10, SEQ ID NO:11, SEQ ID NO:12, SEQ ID NO:13 or SEQ ID NO:14.

In another aspect, an anti-PCSK9 antibody is provided, wherein the antibody comprises a VH as in any of the embodiments provided above, and a VL as in any of the embodiments provided above. In one embodiment, the antibody comprises the VH and VL sequences in SEQ ID NO:15 and SEQ ID NO:18, respectively, including post-translational modifications of those sequences. In one embodiment, the antibody comprises the VH and VL sequences in SEQ ID NO:15 and SEQ ID NO:19, respectively, including post-translational modifications of those sequences. In one embodiment, the antibody comprises the VH and VL sequences in SEQ ID NO:27 and SEQ ID NO:20, respectively, including post-translational modifications of those sequences. In one embodiment, the antibody comprises the VH and VL sequences in SEQ ID NO:16 and SEQ ID NO:21, respectively, including post-translational modifications of those sequences. In one embodiment, the antibody comprises the VH and VL sequences in SEQ ID NO:17 and SEQ ID NO:22, respectively, including post-translational modifications of those sequences. In one embodiment, the antibody comprises the VH and VL sequences in SEQ ID NO:27 and SEQ ID NO:23, respectively, including post-translational modifications of those sequences.

In certain embodiments, functional epitopes can be mapped by combinatorial alanine scanning. In this process, a combinatorial alanine-scanning strategy can be used to identify amino acids in the PCSK9 protein that are necessary for interaction with anti-PCSK9 antibodies. In certain embodiments, the epitope is conformational and crystal structure of anti-PCSK9 antibody Fab fragment bound to PCSK9 may be employed to identify the epitopes. In one aspect, the invention provides an antibody that binds to the same epitope as any of the anti-PCSK9 antibody provided herein. For example, in certain embodiments, an antibody is provided that binds to the same epitope as an anti-PCSK9 antibody comprising a VH sequence of SEQ ID NO:15 and a VL sequence of SEQ ID NO:19. In certain embodiments, an antibody is provided that binds to the same epitope as an anti-PCSK9 antibody comprising a VH sequence of SEQ ID NO:27 and a VL sequence of SEQ ID NO:20. In certain embodiments, an antibody is provided that binds to the same epitope as an anti-PCSK9 antibody comprising a VH sequence of SEQ ID NO:16 and a VL sequence of SEQ ID NO:21. In certain embodiments, an antibody is provided that binds to the same epitope as an anti-PCSK9 antibody comprising a VH sequence of SEQ ID NO:17 and a VL sequence of SEQ ID NO:22. In certain embodiments, an antibody is provided that binds to the same epitope as an anti-PCSK9 antibody comprising a VH sequence of SEQ ID NO:27 and a VL sequence of SEQ ID NO:23.

In one aspect, the invention provides an anti-PCSK9 antibody, or antigen binding fragment thereof, that binds to human PCSK9 competitively with any one of the antibodies described herein. In certain embodiments, competitive binding may be determined using an ELISA assay. For example, in certain embodiments, an antibody is provided that binds to PCSK9 competitively with an anti-PCSK9 antibody comprising a VH sequence of SEQ ID NO:15 and a VL sequence of SEQ ID NO:19. In certain embodiments, an antibody is provided that binds to PCSK9 competitively with an anti-PCSK9 antibody comprising a VH sequence of SEQ ID NO:27 and a VL sequence of SEQ ID NO:20. In certain embodiments, an antibody is provided that binds to PCSK9 competitively with an anti-PCSK9 antibody comprising a VH sequence of SEQ ID NO:16 and a VL sequence of SEQ ID NO:21. In certain embodiments, an antibody is provided that binds to PCSK9 competitively with an anti-PCSK9 antibody comprising a VH sequence of SEQ ID NO:17 and a VL sequence of SEQ ID NO:22. In certain embodiments, an antibody is provided that binds to PCSK9 competitively with an anti-PCSK9 antibody comprising a VH sequence of SEQ ID NO:27 and a VL sequence of SEQ ID NO:23.

In certain embodiments, an antibody is provided that binds to an epitope within a fragment of PCSK9 as described herein. In certain embodiments, an antibody is provided that binds to an epitope within a fragment of PCSK9 comprising amino acids 376 to 379 of human PCSK9 amino acid sequence of SEQ ID NO:24. In certain embodiments, the functional and/or structural epitope of an antibody according to this invention includes residue D238 of human PCSK9. In certain embodiments, the functional and/or structural epitope of an antibody according to this invention includes residue A239 of human PCSK9. In certain embodiments, the functional and/or structural epitope of an antibody according to this invention includes residues D238 and A239 of human PCSK9. In certain embodiments, the functional and/or structural epitope of an antibody according to this invention includes residue E366 of human PCSK9. In certain embodiments, the functional and/or structural epitope of an antibody according to this invention includes residue D367 of human PCSK9. In certain embodiments, the functional and/or structural epitope of an antibody according to this invention includes residues E366 and D367 of human PCSK9. In certain embodiments, the functional and/or structural epitope of an antibody according to this invention includes residue H391 of human PCSK9. In certain embodiments, the functional and/or structural epitope of an antibody according to this invention includes residues E366, D367 and H391 of human PCSK9. According to another embodiment, the functional and/or structural epitope of an antibody according to this invention includes residues A239 and H391 of human PCSK9. In certain embodiments, the functional and/or structural epitope of includes one or more of residues A239, A341, E366, D367 and H391 of human PCSK9. In certain embodiments, the functional and/or structural epitope of includes one or more of residues near A239, A341, E366, D367 and H391 of human PCSK9. In certain embodiments, the functional and/or structural epitope of an antibody according to this invention comprises (i) at least one residue selected from the group consisting of R194 and E195, (ii) at least one residue selected from the group consisting of D238 and A239, (iii) at least one residue selected from the group consisting of A341 and Q342, and (iv) at least one residue selected from the group consisting of E366, D367, 1369, 5376, T377, C378, F379, 5381 and H391, of human PCSK9. In certain embodiments, the functional and/or structural epitope comprises one, two, three, four, five, six, seven, eight, nine, ten, eleven, twelve, thirteen, fourteen or all of the following residues: R194, E195, D238, A239, A341, Q342, E366, D367, 1369, 5376, T377, C378, F379, 5381 and H391 of human PCSK9.

In a further aspect of the invention, an anti-PCSK9 antibody according to any of the above embodiment is a monoclonal antibody, including a chimeric, humanized or human antibody. In one embodiment, an anti-PCSK9 antibody is an antibody fragment, e.g., a Fv, Fab, Fab′, scFv, diabody, or F(ab′)₂ fragment. In another embodiment, the antibody is a full length antibody, e.g., an intact IgG₁ antibody or other antibody class or isotype as defined herein.

In a further aspect, an anti-PCSK9 antibody according to any of the above embodiments may incorporate any of the features, singly or in combination, as described in Sections 1-7 below:

1. Antibody Affinity

In certain embodiments, an antibody provided herein has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ 5⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for PCSK9 and the other is for any other antigen. In certain embodiments, bispecific antibodies may bind to two different epitopes of PCSK9. Bispecific antibodies may also be used to localize cytotoxic agents to cells which express PCSK9. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

The antibody or fragment herein also includes a “Dual Acting FAb” or “DAF” comprising an antigen binding site that binds to PCSK9 as well as another, different antigen (see, e.g., US 2008/0069820).

7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody.

Any combination of deletion, insertion, and substitution can be made to arrive at the final construct, provided that the final construct possesses the desired characteristics, e.g., antigen-binding.

a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “conservative substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (G1cNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). Clq binding assays may also be carried out to confirm that the antibody is unable to bind Clq and hence lacks CDC activity. See, e.g., Clq and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S, and M. J. Glennie, Blood 103:2738-2743 (2004)). FcRn binding and in vivo clearance/half life determinations can also be performed using methods known in the art (see, e.g., Petkova, S. B. et al., Int'l Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) Clq binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

B. Recombinant Methods and Compositions

Anti-PCSK9 antibodies described herein may be produced using recombinant methods and compositions, e.g., as described in U.S. Pat. No. 4,816,567. In one embodiment, isolated nucleic acid encoding an anti-PCSK9 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., YO, NSO, Sp20 cell). In one embodiment, a method of making an anti-PCSK9 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-PCSK9 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.). After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TR1 cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as YO, NSO and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, N.J.), pp. 255-268 (2003).

C. Assays

Anti-PCSK9 antibodies provided herein may be identified, screened for, or characterized for their physical/chemical properties and/or biological activities by various assays known in the art.

1. Binding Assays and Other Assays

In one aspect, an anti-PCSK9 antibody of the invention is tested for its PCSK9 binding activity, e.g., by known methods such as ELISA, Western blot, etc. Numerous types of competitive binding assays can be used to determine if an anti-PCSK9 antibody competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabelled test antigen binding protein and a labeled reference antigen binding protein. Competitive inhibition is measured by determining the amount of label bound to the solid surface or cells in the presence of the test antigen binding protein. Usually the test antigen binding protein is present in excess. Antigen binding proteins identified by competition assay (competing antigen binding proteins) include antigen binding proteins binding to the same epitope as the reference antigen binding proteins and antigen binding proteins binding to an adjacent epitope sufficiently proximal to the epitope bound by the reference antigen binding protein for steric hindrance to occur. Additional details regarding methods for determining competitive binding are provided in the examples herein. Usually, when a competing antigen binding protein is present in excess, it will inhibit (e.g., reduce) specific binding of a reference antigen binding protein to a common antigen by at least 40-45%, 45-50%, 50-55%, 55-60%, 60-65%, 65-70%, 70-75% or 75% or more. In certain embodiments, binding is inhibited by at least 80-85%, 85-90%, 90-95%, 95-97%, or 97% or more.

In one aspect of the invention, competition assays may be used to identify an antibody that competes with anti-PCSK9 antibody 508.20.04, 508.20.06, 508.20.28, 508.20.33 or 508.20.84 for binding to PCSK9. In certain embodiments, such a competing antibody binds to the same epitope (e.g., a linear or a conformational epitope) that is bound by anti-PCSK9 antibody 508.20.04, 508.20.06, 508.20.28, 508.20.33 and/or 508.20.84. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized PCSK9 is incubated in a solution comprising a first labeled antibody that binds to PCSK9 (e.g., anti-PCSK9 antibody 508.20.04, 508.20.06, 508.20.28, 508.20.33 or 508.20.84) and a second unlabeled antibody that is being tested for its ability to compete with the first antibody for binding to PCSK9. The second antibody may be present in a hybridoma supernatant. As a control, immobilized PCSK9 is incubated in a solution comprising the first labeled antibody but not the second unlabeled antibody. After incubation under conditions permissive for binding of the first antibody to PCSK9, excess unbound antibody is removed, and the amount of label associated with immobilized PCSK9 is measured. If the amount of label associated with immobilized PCSK9 is substantially reduced in the test sample relative to the control sample, then that indicates that the second antibody is competing with the first antibody for binding to PCSK9. See Harlow and Lane (1988) Antibodies: A Laboratory Manual ch.14 (Cold Spring Harbor Laboratory, Cold Spring Harbor, N.Y.).

2. Activity Assays

In one aspect, assays are provided for identifying anti-PCSK9 antibodies thereof having biological activity. Biological activity of the anti-PCSK9 antibodies may include, e.g., blocking, antagonizing, suppressing, interfering, modulating and/or reducing one or more biological activities of PCSK9. Antibodies having such biological activity in vivo and/or in vitro are provided.

In certain embodiments, anti-PCSK9 antibody binds human PCSK9 and prevents interaction with the LDLR. In certain embodiments, anti-PCSK9 antibody binds specifically to human PCSK9 and/or substantially inhibits binding of human PCSK9 to LDLR by at least about 20%-40%, 40-60%, 60-80%, 80-85%, or more (for example, by measuring binding in an in vitro competitive binding assay). In certain embodiments, the invention provides isolated anti-PCSK9 antibodies which specifically bind to PCSK9 and which antagonize the PCSK9-mediated effect on LDLR levels when measured in vitro using the LDLR down regulation assay in HepG2 cells disclosed herein.

D. Immunoconjugates

The invention also provides immunoconjugates comprising an anti-PCSK9 antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SLAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

E. Methods and Compositions for Diagnostics and Detection

In certain embodiments, any of the anti-PCSK9 antibodies provided herein is useful for detecting the presence of PCSK9 in a biological sample. The term “detecting” as used herein encompasses quantitative or qualitative detection. In certain embodiments, a biological sample is blood, serum or other liquid samples of biological origin. In certain embodiments, a biological sample comprises a cell or tissue.

In one embodiment, an anti-PCSK9 antibody for use in a method of diagnosis or detection is provided. In a further aspect, a method of detecting the presence of PCSK9 in a biological sample is provided. In certain embodiments, the method comprises detecting the presence of PCSK9 protein in a biological sample. In certain embodiments, PCSK9 is human PCSK9. In certain embodiments, the method comprises contacting the biological sample with an anti-PCSK9 antibody as described herein under conditions permissive for binding of the anti-PCSK9 antibody to PCSK9, and detecting whether a complex is formed between the anti-PCSK9 antibody and PCSK9. Such method may be an in vitro or in vivo method. In one embodiment, an anti-PCSK9 antibody is used to select subjects eligible for therapy with an anti-PCSK9 antibody, e.g. where PCSK9 or LDL-cholesterol is a biomarker for selection of patients.

Exemplary disorders that may be diagnosed using an antibody of the invention include cholesterol related disorders (which includes “serum cholesterol related disorders”), including any one or more of the following: hypercholesterolemia, heart disease, metabolic syndrome, diabetes, coronary heart disease, stroke, cardiovascular diseases, Alzheimers disease and generally dyslipidemias, which can be manifested, for example, by an elevated total serum cholesterol, elevated LDL, elevated triglycerides, elevated very low density lipoprotein (VLDL), and/or low HDL. In one aspect, the invention provides a method for treating or preventing hypercholesterolemia, and/or at least one symptom of dyslipidemia, atherosclerosis, cardiovascular disease (CVD) or coronary heart disease, in an individual comprising administering to the individual an effective amount of anti-PCSK9 antibody. In certain embodiments, the invention provides an effective amount of an anti-PCSK9 antibody for use in treating or preventing hypercholesterolemia, and/or at least one symptom of dyslipidemia, atherosclerosis, CVD or coronary heart disease, in a subject. The invention further provides the use of an effective amount of an anti-PCSK9 antibody that antagonizes extracellular or circulating PCSK9 in the manufacture of a medicament for treating or preventing hypercholesterolemia, and/or at least one symptom of dyslipidemia, atherosclerosis, CVD or coronary heart disease, in an individual.

In certain embodiments, labeled anti-PCSK9 antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, f3-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

F. Pharmaceutical Formulations

This invention also encompasses compositions, including pharmaceutical formulations, comprising an anti-PCSK9 antibody, and polynucleotides comprising sequences encoding an anti-PCSK9 antibody. In certain embodiments, compositions comprise one or more antibodies that bind to PCSK9, or one or more polynucleotides comprising sequences encoding one or more antibodies that bind to PCSK9. These compositions may further comprise suitable carriers, such as pharmaceutically acceptable excipients including buffers, which are well known in the art.

Pharmaceutical formulations of an anti-PCSK9 antibody as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH2O (HYLENEX®, Baxter International, Inc.). Certain exemplary sHASEGPs and methods of use, including rHuPH2O, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide statin. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

G. Therapeutic Methods and Compositions

Any of the anti-PCSK9 antibodies provided herein may be used in therapeutic methods.

In one aspect, an anti-PCSK9 antibody for use as a medicament is provided. In another aspect, an anti-PCSK9 antibody for use in treating conditions associated with cholesterol related disorder is provided. In certain embodiments, an anti-PCSK9 antibody for use in treating conditions associated with elevated level of LDL-cholesterol is provided. In certain embodiments, an anti-PCSK9 antibody for use in a method of treatment is provided. In certain embodiments, the invention provides an anti-PCSK9 antibody for use in a method of treating an individual having conditions associated with elevated level of LDL-cholesterol comprising administering to the individual an effective amount of the anti-PCSK9 antibody. In certain embodiments, the methods and uses described herein further comprise administering to the individual an effective amount of at least one additional therapeutic agent, e.g., statin. In certain embodiments, the invention provides an anti-PCSK9 antibody for use in reducing LDL-cholesterol level in a subject. In further embodiments, the invention provides an anti-PCSK9 antibody for use in lowering serum LDL-cholesterol level in a subject. In certain embodiments, the invention provides an anti-PCSK9 antibody for use in increasing availability of LDLR in a subject. In certain embodiments, the invention provides an anti-PCSK9 antibody for use in inhibiting binding of PCSK9 to LDLR in a subject. In certain embodiments, the invention provides an anti-PCSK9 antibody for use in a method of reducing LDL-cholesterol level in an individual comprising administering to the individual an effective of the anti-PCSK9 antibody to reduce the LDL-cholesterol level. In certain embodiments, the invention provides an anti-PCSK9 antibody for use in a method of lowering serum LDL-cholesterol level in an individual comprising administering to the individual an effective of the anti-PCSK9 antibody to lower the serum LDL-cholesterol level. In certain embodiments, the invention provides an anti-PCSK9 antibody for use in a method of increasing availability of LDLR in an individual comprising administering to the individual an effective of the anti-PCSK9 antibody to increase availability of LDLR. In certain embodiments, the invention provides an anti-PCSK9 antibody for use in a method of inhibiting binding of PCSK9 to LDLR in an individual comprising administering to the individual an effective of the anti-PCSK9 antibody to inhibit the binding of PCSK9 to LDLR. An “individual” according to any of the above embodiments is preferably a human.

In a further aspect, the invention provides for the use of an anti-PCSK9 antibody in the manufacture or preparation of a medicament. In one embodiment, the medicament is for treatment of cholesterol related disorder. In certain embodiments, the cholesterol related disorder is hypercholesterolemia. In another embodiment, the medicament is for use in a method of treating hypercholesterolemia comprising administering to an individual having hypercholesterolemia an effective amount of the medicament.

In certain embodiments, the disorder treated is any disease or condition which is improved, ameliorated, inhibited or prevented by removal, inhibition or reduction of PCSK9 activity. In certain embodiments, diseases or disorders that are generally addressable (either treatable or preventable) through the use of statins can also be treated. In certain embodiments, disorders or disease that can benefit from the prevention of cholesterol synthesis or increased LDLR expression can also be treated by anti-PCSK9 antibodies of the present invention. In certain embodiments, individuals treatable by the anti-PCSK9 antibodies and therapeutic methods of the invention include individuals indicated for LDL apheresis, individuals with PCSK9-activating mutations (gain of function mutations, “GOF”), individuals with heterozygous Familial Hypercholesterolemia (heFH), individuals with primary hypercholesterolemia who are statin intolerant or statin uncontrolled, and individuals at risk for developing hypercholesterolemia who may be preventably treated. Other indications include dyslipidemia associated with secondary causes such as Type 2 diabetes mellitus, cholestatic liver diseases (primary biliary cirrhosis), nephrotic syndrome, hypothyroidism, obesity, and the prevention and treatment of atherosclerosis and cardiovascular diseases.

In certain embodiments, the methods and uses described herein further comprises administering to the individual an effective amount of at least one additional therapeutic agent, e.g., statin. In certain embodiments, the additional therapeutic agent is for preventing and/or treating atherosclerosis and/or cardiovascular diseases. In certain embodiment, the additional therapeutic agent is for use in a method of reducing the risk of recurrent cardiovascular events. In certain embodiments, the additional therapeutic agent is for elevating the level of HDL-cholesterol in a subject.

In a further aspect, the invention provides pharmaceutical formulations comprising any of the anti-PCSK9 antibodies provided herein, e.g., for use in any of the above therapeutic methods. In one embodiment, a pharmaceutical formulation comprises any of the anti-PCSK9 antibodies provided herein and a pharmaceutically acceptable carrier. In another embodiment, a pharmaceutical formulation comprises any of the anti-PCSK9 antibodies provided herein and at least one additional therapeutic agent, e.g., statin.

Antibodies of the invention can be used either alone or in combination with other agents in a therapy. For instance, an antibody of the invention may be co-administered with at least one additional therapeutic agent. In certain embodiments, such additional therapeutic agent elevates the level of LDLR. In certain embodiments, an additional therapeutic agent is a LDL-cholesterol lowering drugs such as statin, e.g., atorvastatin, fluvastatin, lovastatin, mevastatin, pitavastatin, pravastatin, rosuvastatin, simvastatin, or any combination thereof, e.g., VYTORIN®, ADVICOR® or SIMCOR®. In certain embodiments, an additional therapeutic agent is a HDL-cholesterol raising drugs.

Such combination therapies noted above encompass combined administration (where two or more therapeutic agents are included in the same or separate formulations), and separate administration, in which case, administration of the anti-PC SK9 antibody of the invention can occur prior to, simultaneously, and/or following, administration of the additional therapeutic agent and/or adjuvant.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Anti-PC SK9 antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered.

In certain embodiments, a flat-fixed dosing regimen is used to administer anti-PCSK9 antibody to an individual. Depending on the type and severity of the disease an exemplary flat-fixed dosage might range from 10 to 1000 mg of anti-PCSK9 antibody. One exemplary dosage of the antibody would be in the range from about 10 mg to about 600 mg. Another exemplary dosage of the antibody would be in the range from about 100 mg to about 600 mg. In certain embodiments, 150 mg, 300 mg, or 600 mg of anti-PCSK9 antibody is administered to an individual. However, other dosage regimens may be useful. The progress of this therapy is easily monitored by conventional techniques and assays.

It is understood that any of the above formulations or therapeutic methods may be carried out using an immunoconjugate of the invention in place of or in addition to an anti-PCSK9 antibody.

H. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an anti-PCSK9 antibody of the invention. The label or package insert indicates that the composition is used for treating the condition of choice. Moreover, the article of manufacture may comprise (a) a first container with a composition contained therein, wherein the composition comprises an anti-PCSK9 antibody of the invention; and (b) a second container with a composition contained therein, wherein the composition comprises a further cytotoxic or otherwise therapeutic agent. In certain embodiments, the second container comprises a second therapeutic agent, wherein the second therapeutic agent is a cholesterol-lowering drug of the “statin” class. In certain embodiments, the statin is and/or comprises atorvastatin (e.g., LIPITOR® or Torvast), fluvastatin (e.g., LESCOL), lovastatin (e.g., MEVACOR®, ALTOCOR™, or ALTOPREV), mevastatin (pitavastatin (e.g., LIVALO® or PITAVA®), pravastatin (e.g., PRAVACHOL®, SELEKTINE®, LIPOSTAT®), rosuvastatin (e.g., CRESTOR®), simvastatin (e.g., ZOCOR®, LIPEX®), or any combination thereof, e.g., VYTORIN®, ADVICOR® or SIMCOR®.

The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to an anti-PCSK9 antibody.

### III. EXAMPLES

The following are examples of methods and compositions of the invention. It is understood that various other embodiments may be practiced, given the general description provided above.

### Example 1

### Generation of Anti-PCSK9 Antibodies

Residue numbers are according to Kabat (Kabat et al., Sequences of proteins of immunological interest, 5th Ed., Public Health Service, National Institutes of Health, Bethesda, Md. (1991)).

Library Sorting and Screening to Identify Anti-PCSK9 Antibodies

Biotinylated human PCSK9 generated in-house was used as antigen for library sorting.

The phage libraries were sorted five rounds against biotinylated PCSK9 in solution phase. For the first round of sorting, 20 μg/mL biotinylated PCSK9 was added to antibody phage libraries VH (see, e.g., Lee et al., J. Immunol. Meth. 284:119-132, 2004) and VH/VL (see Liang et al., JMB. 366: 815-829, 2007) pre-blocked with phage blocking buffer PBST (phosphate-buffered saline (PBS) and 1% (w/v) bovine serum albumin (BSA) and 0.05% (v/v) Tween 20) and incubated overnight at room temperature. The following day 120 μl of PBST/BSA pre-absorbed DYNABEADS® MyOne™ Streptavidin T1 (Invitrogen, Carlsbad, Calif.) was added to each library and incubated for 1 hour at room temperature. The beads were then washed three times with PBT (PBS with 0.05% Tween 20), and bound phage were eluted with 1 mL 50 mM HCl and 500 mM NaCl for 30 minutes and neutralized with 4004 of 1 M Tris base (pH7.5). Recovered phages were amplified in E. coli XL-1 Blue cells. During the subsequent selection rounds, incubation of antibody phage with the biotinylated PCSK9 was reduced to 2-3 hours, and the phage bound antigen was captured for 30 minutes on neutravidin-coated (Catalog #89890, 10 μg/ml, Fisher Scientific, Waltham, Mass.) or streptavidin-coated (Catalog #21125, 10 μg/ml, Fisher Scientific, Waltham, Mass.) Nunc 96 well Maxisorp immunoplates. The stringency of plate washing was gradually increased.

After 5 rounds of panning, significant enrichment was observed. 96 clones were picked each from VH and VH/VL library sorting to determine whether they specifically bound to human PCSK9. The variable regions of these clones were PCR sequenced to identify unique sequence clones. Unique phage antibodies that bind human PCSK9 at least 5× above background were chosen and reformatted to full length IgGs for evaluation in in vitro cell assay.

Clones of interest were reformatted into IgGs by cloning VL and VH regions of individual clones into the LPG3 and LPG4 vector respectively, transiently expressing in mammalian CHO cells, and purifying with a protein A column.

Construct Libraries for Affinity Improvement of Clones Derived from the VH Library

Phagemid pWO703 (derived from phagemid pV0350-2b (Lee et al., J. Mol. Biol. 340, 1073-1093 (2004)), containing stop codon (TAA) in all CDR-L3 positions and displaying monovalent Fab on the surface of M13 bacteriophage served as the library template for grafting heavy chain variable domains (VH) of clones of interest from the VH library for affinity maturation. Both hard and soft randomization strategies were used for affinity maturation. For hard randomization, one light chain library with selected positions of the three light chain CDRs was randomized using amino acids designed to mimic natural human antibodies and the designed DNA degeneracy was as described in Lee et al. (J. Mol. Biol. 340, 1073-1093 (2004)). For soft randomization, residues at positions 91-96 of CDR-L3, 30-33, 35 of CDR-H1, 50, 52, 53-54, 56, and 58 of CDR-H2, 95-100, 100A, and 100C of CDR-H3, were targeted; and three different combinations of CDR loops, H1/L3, H2/L3, and H3/L3, were selected for randomization. To achieve the soft randomization conditions, which introduced the mutation rate of approximately 50% at the selected positions, the mutagenic DNA was synthesized with 70-10-10-10 mixtures of bases favoring the wild type nucleotides (Gallop et al., Journal of Medicinal Chemistry 37:1233-1251 (1994)).

Phage Sorting Strategy to Generate Affinity Improvement

For affinity improvement selection, phage libraries were subjected to five rounds of solution sorting with increasing stringency. For the first round of solution sorting, 3 O.D./ml in 1% BSA and 0.05% Tween 20 of phage input were incubated with 100 nM biotinylated PCSK9 (the concentration is based on parental clone phage IC50 value) in 100 μl buffer containing 1% Superblock (Pierce Biotechnology) and 0.05% Tween 20 for 2 hours at room temperature. The mixture was further diluted 10× with 1% Superblock, and 100 μl/well was applied to neutravidin-coated wells (10 m/ml) for 30 minutes at room temperature with gentle shaking. The wells were washed with PBS-0.05% Tween 20 ten times. To determine background binding, control wells containing phage were captured on neutravidin-coated plates. Bound phage was eluted with 150 μl/well 50 mM HCl, 500 mM KCl for 30 minutes, and subsequently neutralized by 50 μl/well of 1M Tris pH8, titered, and propagated for the next round. Four more rounds of solution sorting were carried out together with increasing selection stringency. The first couple of rounds were for on-rate selection by decreasing biotinylated target protein concentration from 100 nM to 1 nM, and the last two rounds were for off-rate selection by adding excess amounts of non-biotinylated target protein (300 to 1000 fold more) to compete off weaker binders at room temperature.

High Throughput Affinity Screening ELISA (Single Spot Competition)

Colonies were picked from the fifth round of screening. Colonies were grown overnight at 37° C. in 150 μl/well of 2YT media with 50 m/ml carbenicillin and 1E10/ml KO7 in 96-well plate (Falcon). From the same plate, a colony of XL-1 infected parental phage was picked as control. 96-well Nunc Maxisorp plates were coated with 100 μl/well of neutravidin (10 m/ml) in PBS at 4° C. overnight. The plates were blocked with 150 μl of 1% BSA and 0.05% Tween 20 in PBS for 1 hour.

35 μl of the phage supernatant was diluted with 35 μl of ELISA (enzyme linked immunosorbent assay) buffer (PBS with 0.5% BSA, 0.05% Tween 20) with or without 15 nM PCSK9 and let incubate for 1 hour at room temperature in an F plate (NUNC). 35 μl of 3 μg/ml biotinylated-PCSK9 was then added to each well and incubated for 15 minutes at room temperature. 95 μl of mixture was transferred side by side to the neutravidin coated plates. The plate was gently shaken for 15 min to allow the capture of biotinylated-PCSK9 bound phage to the plate. The plate was washed ten times with PBS-0.05% Tween 20. The binding was quantified by adding horseradish peroxidase (HRP)-conjugated anti-M13 antibody in ELISA buffer (1:2500) and incubated for 30 minutes at room temperature. The plates were washed with PBS-0.05% Tween 20 ten times. Next, 100 μl/well of a 1:1 ratio of 3,3′,5,5′-tetramethylbenzidine (TMB) Peroxidase substrate and Peroxidase Solution B (H₂O₂) (Kirkegaard-Perry Laboratories (Gaithersburg, Md.)) was added to the well and incubated for 5 minutes at room temperature. The reaction was stopped by adding 100 μl 10.1M Phosphoric Acid (H₃PO₄) to each well and allowed to incubate for 5 minutes at room temperature. The O.D. (optical density) of the yellow color in each well was determined using a standard ELISA plate reader at 450 nm. The O.D. reduction (%) was calculated by the following equation:

OD₄₅₀ₙₘreduction(%)=[(OD₄₅₀ₙₘ of wells with competitor)/(OD₄₅₀ₙₘ of well with no competitor)]*100

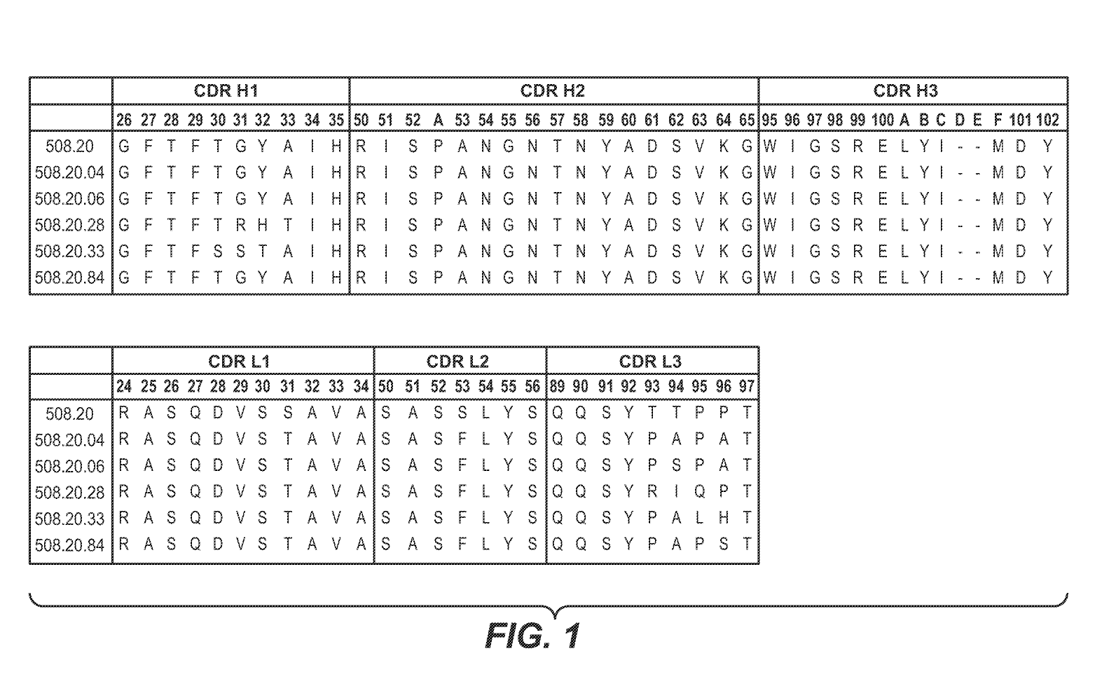

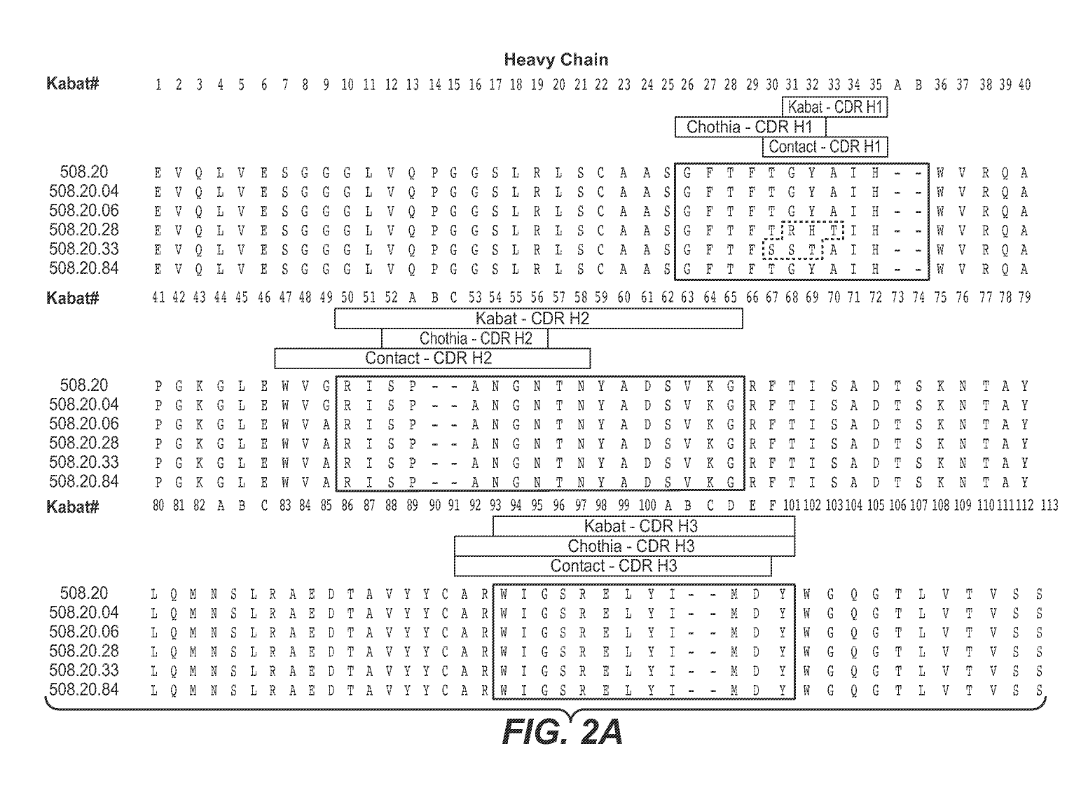

In comparison to the OD₄₅₀ₙₘ reduction (%) of the well of parental phage (100%), clones that had the OD₄₅₀ₙₘ reduction (%) lower than 50% were picked for sequence analysis. Unique clones were selected for phage preparation to determine binding affinity (phage IC₅₀) against PCSK9 by comparison to parental clone. Then the most affinity-improved clones were reformatted into human IgG₁ for antibody production and further BIAcore binding kinetic analysis and other in vitro or in vivo assay. See FIGS. 1 and 2.

### Example 2

### Characterization of Anti-PCSK9 Antibodies by BIAcore

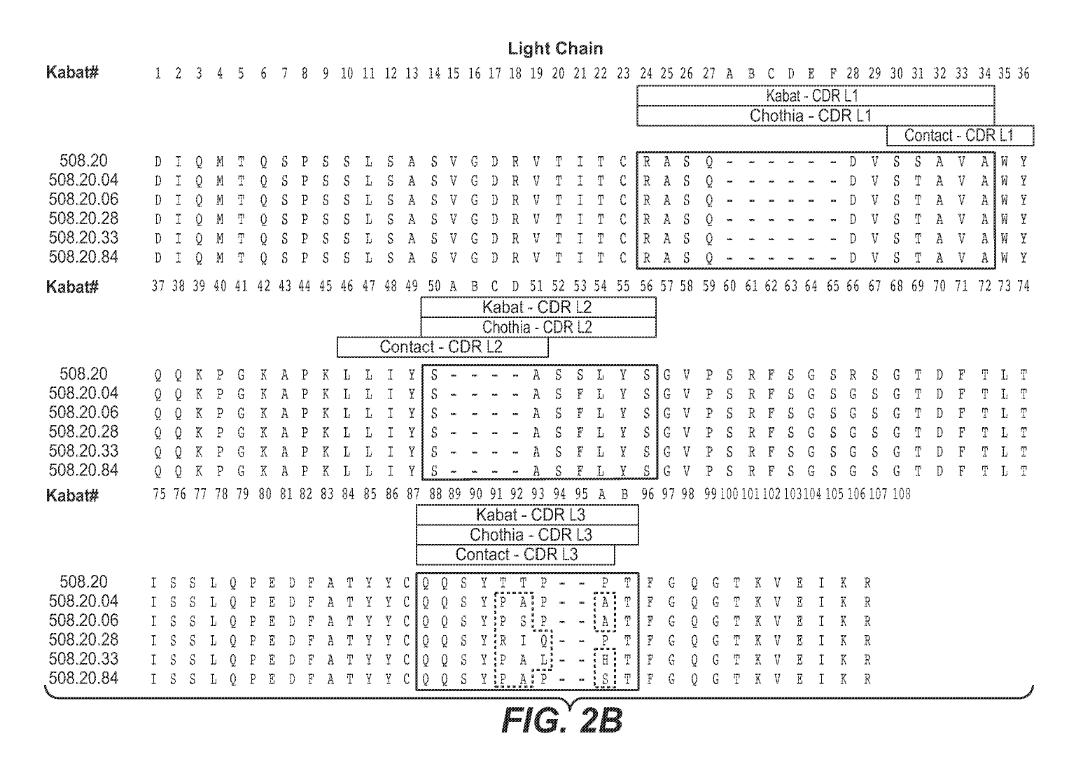

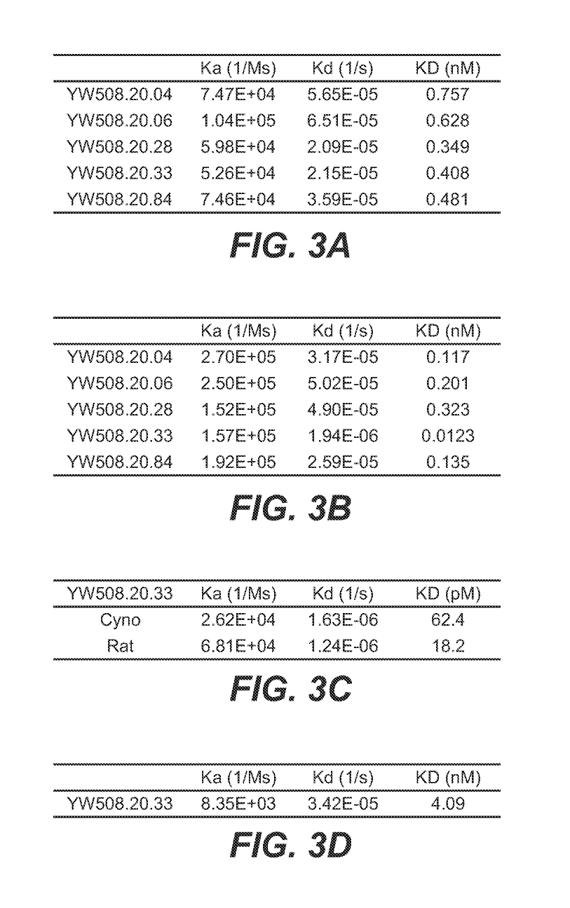

Binding affinities of anti-PCSK9 antibodies were measured by Surface Plasmon Resonance (SRP) using a BIAcore™-3000 instrument. Anti-PCSK9 human antibodies were captured by mouse anti-human Fc antibody (Catalog #BR-1008-39, GE Healthcare, Piscataway, N.J.) coated on CM5 biosensor chips to achieve approximately 200 response units (RU). For kinetics measurements, two-fold serial dilutions (500 nM to 0.245 nM) of human, murine, rhesus, and cyno PCSK9 (Genentech, South San Francisco, Calif.) were injected in PBT buffer (PBS with 0.05% Tween 20) at 25° C. with a flow rate of 30 μl/min. Association rates (kₒₙ) and dissociation rates (k_{off}) were calculated using a simple one-to-one Langmuir binding model (BIAcore Evaluation Software version 3.2). The equilibrium dissociation constant (K_{D}) was calculated as the ratio k_{off}/kₒₙ. See FIG. 3.

### Example 3

### LDLR-PCSK9 Binding Assay

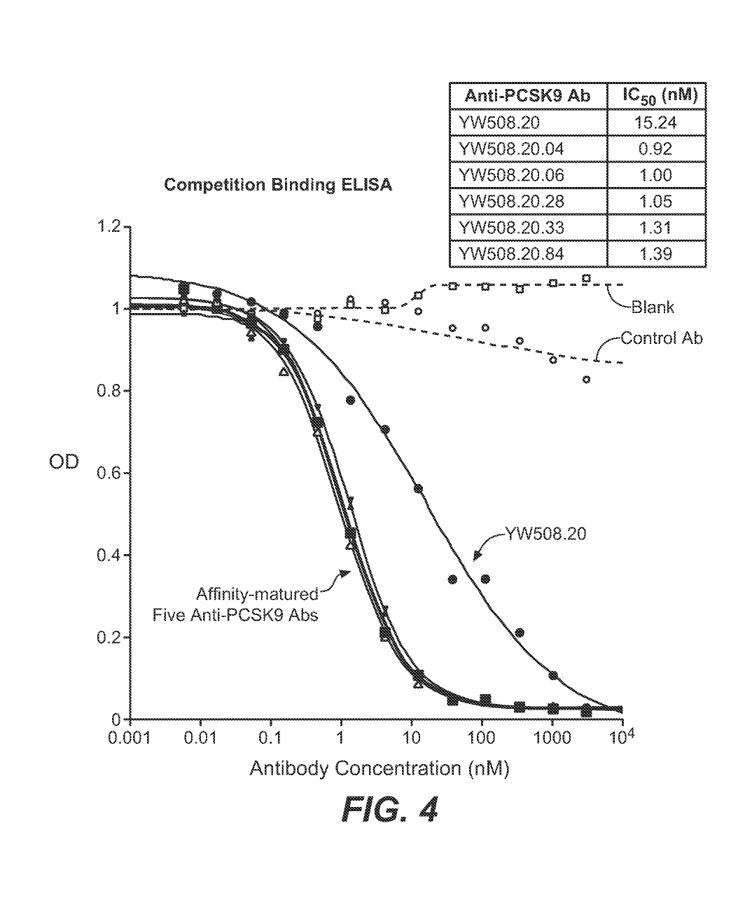

A competition binding ELISA was performed to investigate the activity of anti-PCSK9 antibodies in blocking human PCSK9 binding to LDLR. Briefly, 1 μg/mL of soluble human LDLR extracellular domain (R&D Systems, Minneapolis, Minn.) was coated on 384-well MaxiSorp plate (NALGENE® NUNC™ International, Rochester, N.Y.) at 4° C. overnight. Then 0.25 μg/mL of biotinylated human PCSK9 pre-mixed with different concentrations of anti-PCSK9 antibodies and control antibodies were added to the plate and incubated for 2 hour at room temperature. The binding of PCSK9 to coated LDLR was detected by adding streptavidin-HRP (GE Healthcare) and substrate 3,3′,5,5′-tetramethyl benzidine (TMBE-1000, Moss, Inc., Pasadena, Md.). The binding results (OD) were plotted against antibody concentration and IC₅₀ values were generated using KaleidaGraph software. See FIG. 4.

### Example 4

### Antibodies Prevent LDLR Downregulation on HepG2 Cells

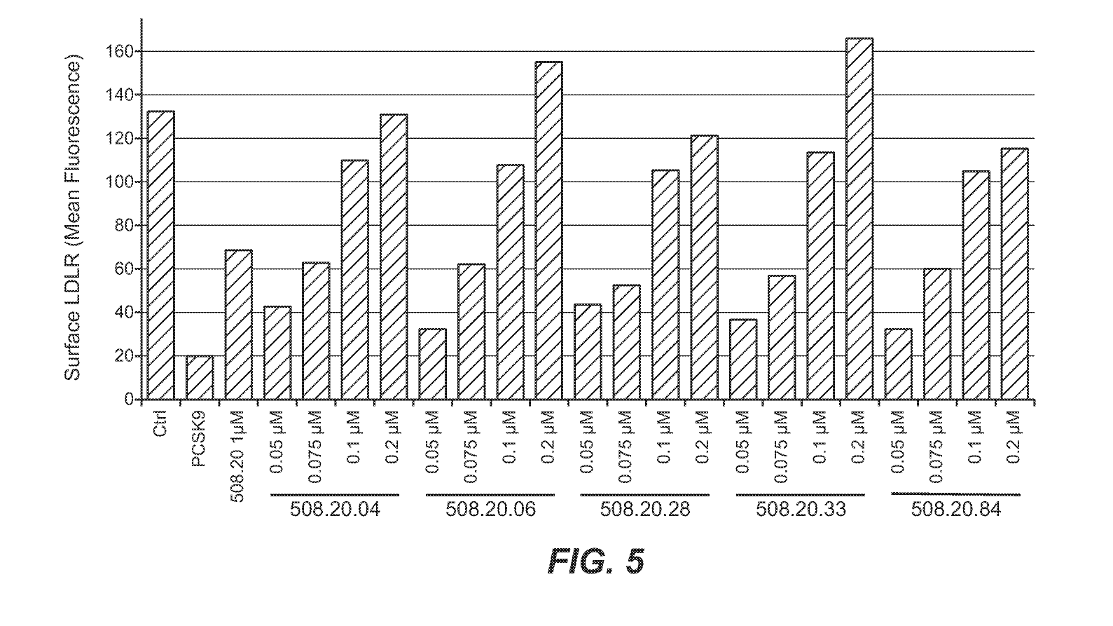

HepG2 cells were seeded at 1×10⁵ into a 48-well plate. The next day, the media was changed to 10% lipoprotein deficient serum (LPDS, Frederick, Md.). The following day, 15 μg/mlPCSK9 plus/minus anti-PCSK9 antibody were added to cells for 4 hours at 37° C. Cells were rinsed with PBS and detached using 2.5 mM EDTA. Cells were incubated with 1:20 anti-LDLR (Progen Biotechnik, Heidelberg, Germany) for 15 minutes, washed with PBS and incubated with 1:200 goat anti-mouse AlexaFluor 488 from Invitrogen (Carlsbad, Calif.) for 15 minutes. Cells were washed and resuspended in PBS plus 10 μg/ml propidium iodide. The samples were then analyzed with a dual laser flow cytometer (FACScan, Becton Dickinson, Franklin Lakes, N.J.). The data suggest all of the anti-PCSK9 antibodies described herein prevent downregulation of LDLR. See FIG. 5.

### Example 5

### LDLR Downregulation in Mouse Liver

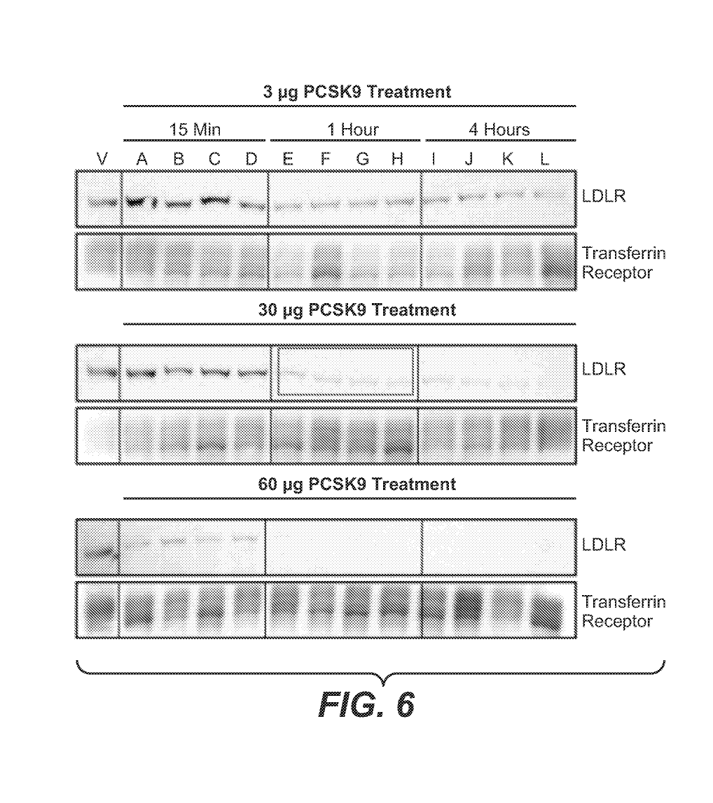

Normal C57/BL6 mice (Charles River, Wilmington, Mass.) were treated with 3, 30 or 60 μg of PCSK9 by I.V. administration. Using the ProteoExtract Native Membrane Protein Extraction Kit from Calbiochem (Gibbstown, N.J.) according to the manufacturer's instructions, liver from each mouse was harvested 15 min, 1 hr or 4 hrs after PCSK9 I.V. administration and proteins extracted. As a control, 5 mice were treated with vehicle only and 8 μg of each liver lysate were pooled for analysis. Lysates were analyzed by SDS-PAGE on 8% tris-gly gel (Invitrogen, Carlsbad, Calif.). Proteins were transferred to nitrocellulose membrane using iBlot (Invitrogen). The membrane was blocked with 5% nonfat milk for 1 hour and then incubated with 1:500 anti-LDLR (Abcam, Cambridge, Mass.) in 5% nonfat milk overnight at 4° C. The next day, the membrane was washed three times with TBS-T, incubated with 1:5000 anti-rabbit HRP (GE Healthcare, Piscataway, N.J.) for 1 hour and washed with TBS-T three times. Proteins were visualized using ECL-Plus (GE Healthcare) and exposed to XAR film (Kodak, Rochester, N.Y.). After an overnight exposure, the membrane was washed with TBS-T, incubated with 1:500 anti-transferrin receptor antibody (Invitrogen) for 1 hour, washed with TBS-T, incubated with 1:5000 anti-mouse HRP (GE Healthcare) for 1 hour, washed with TBS-T and visualized with ECL-Plus. Western blot with anti-LDLR antibody shows that 30 μg of PCSK9 for 1 hour significantly downregulated LDLR levels in mouse liver. See FIG. 6.

### Example 6

### Antibodies Prevent Liver LDLR Downregulation

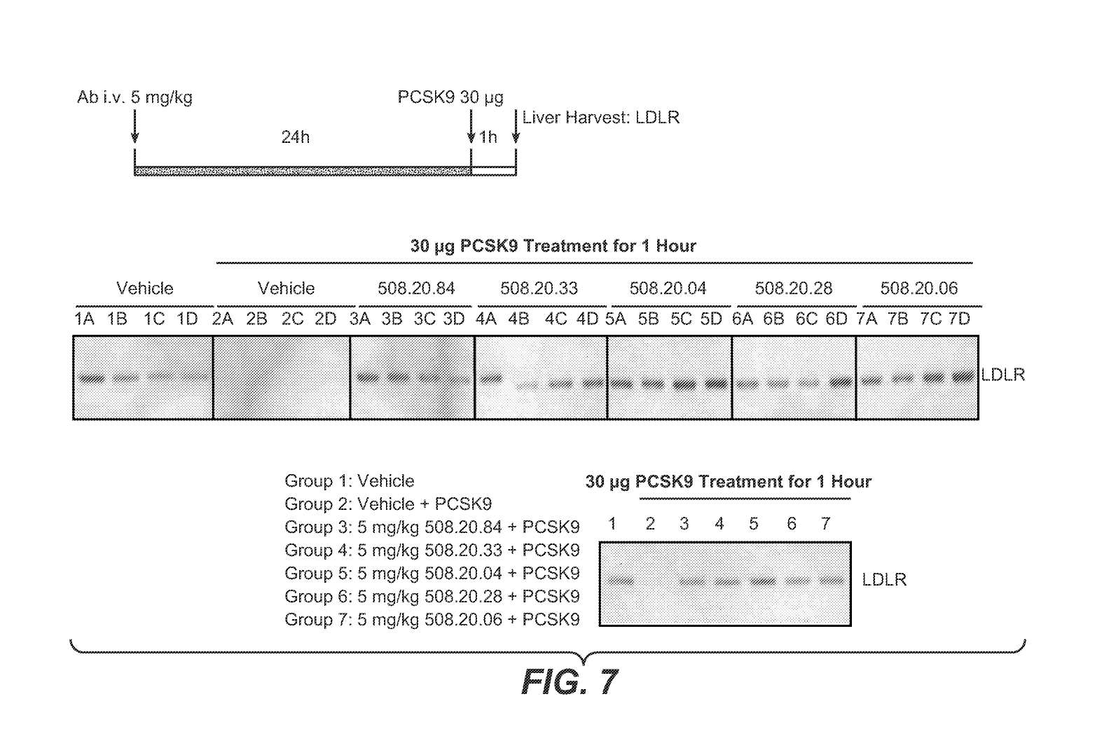

Normal C57/BL6 (Charles River) mice were injected with vehicle or 5 mg/kg anti-PCSK9 antibodies 24 hours prior to treatment with 30 μg PCSK9 for 1 hour. Liver from each mouse was harvested using the ProteoExtract Native Membrane Protein Extraction Kit (Calbiochem) according to the manufacturer's instructions. Lysates were analyzed by SDS-PAGE on 8% bis-tris gel. Proteins were transferred to nitrocellulose membrane using iBlot (Invitrogen). The membrane was blocked with 5% nonfat milk for 1 hour and then incubated with 1:500 anti-LDLR (Abcam) in 5% nonfat milk overnight at 4° C. The next day, the membrane was washed three times with TBS-T, incubated with 1:5000 anti-rabbit HRP (GE Healthcare) for 1 hour and washed three times with TBS-T. Proteins were visualized using ECL-Plus (GE Healthcare) and exposed to XAR film (Kodak). Western blot with anti-LDLR antibody show that all five anti-PCSK9 antibodies (508.20.84, 508.20.33, 508.20.04, 508.20.28, 508.20.06) prevented LDLR downregulation in mouse liver. See FIG. 7.

### Example 7

### Pharmacokinetics of Anti-PCSK9 Antibody

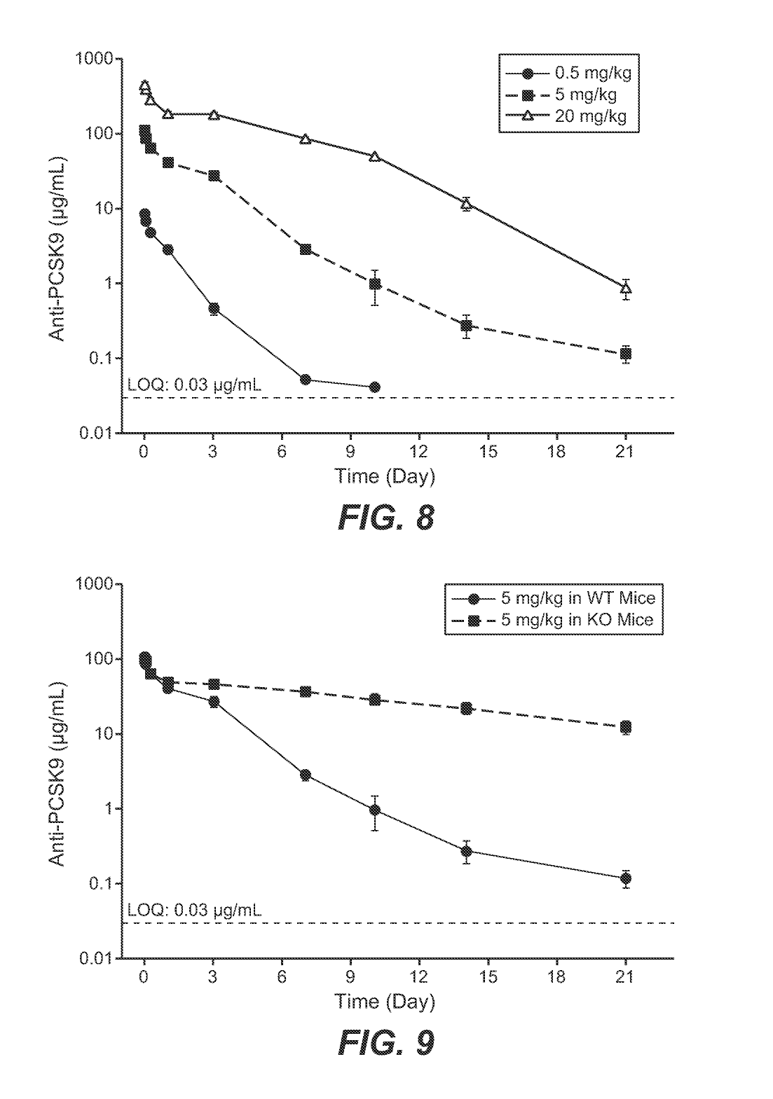

Anti-PCSK9 antibody concentrations in mouse PK study samples were determined using anti-human IgG Fc ELISA. Briefly, donkey anti-human IgG Fc (Jackson ImmunoResearch, West Grove, Pa.) was used to coat assay plates and goat anti-human IgG Fc HRP conjugate (Jackson ImmunoResearch, West Grove, Pa.) was used as detection antibody. The assay was able to measure anti-PCSK9 antibody in up to 10% mouse serum matrix with assay range of 0.31-20 ng/mL. See FIGS. 8 and 9.

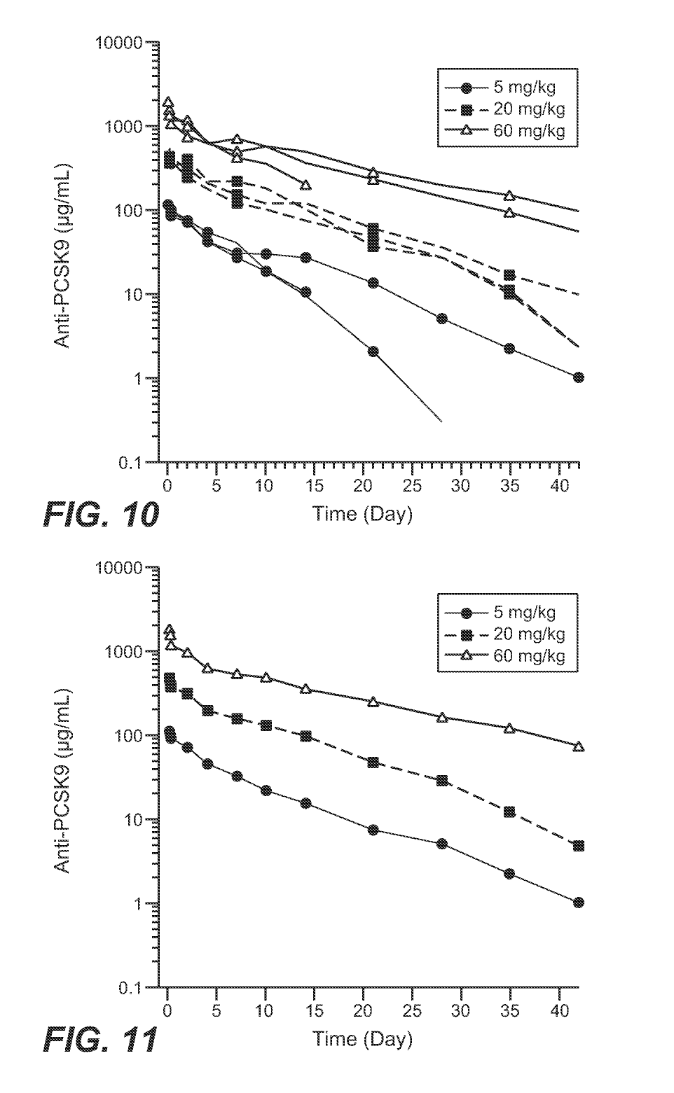

Serum anti-PCSK9 antibody concentrations in cynomolgus monkey PK study samples were determined by anti-PCSK9 antibody ELISA using recombinant human PCSK9 (Genentech, Inc. South San Francisco, Calif.) as capture and goat anti-human IgG (H+ L) HRP as detection antibody. The assay was able to measure anti-PCSK9 antibody in up to 2% cynomolgus monkey serum matrix with assay range of 0.313-50 ng/mL. See FIGS. 10 and 11.

### Example 8

### Antibodies Reduce Serum Cholesterol Level in Mice

Eight weeks old male C57BL/6J mice were purchased commercially from Jackson Laboratory. The mice were on housing for one week at the holding room before the start of the experiment. All mice were pre-bled under anesthesia and total cholesterol levels from the mice were determined using INFINITY™ Cholesterol Reagent (Fisher Diagnostics, Middletown, Va.). The mice were randomized into 6 different groups with the same level of average cholesterol level. All mice received a single dose of 10 mg/kg body weight of either control antibody or anti-PCSK9 antibodies. The mice were bled on day 3, day 7, day 10 and day 15 and serum total cholesterol levels were determined using INFINITY™ Cholesterol Reagent (Fisher Diagnostics, Middletown, Va.).

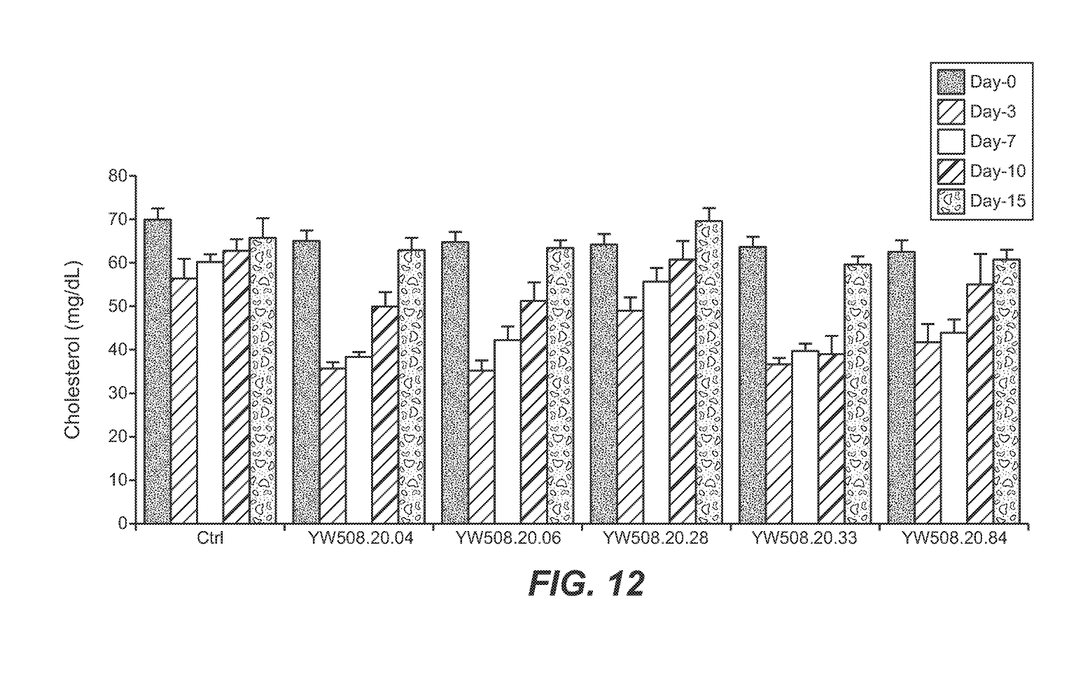

All five anti-PCSK9 antibodies (508.20.04, 508.20.06, 508.20.28, 508.20.33, 508.20.84) showed a reduction in total cholesterol levels when a single dose of 10 mg/kg was administered. The administration of anti-PCSK9 antibody resulted in a significant reduction in total cholesterol level on day 3 and up to day 10 when compared to the mice receiving control antibody. See FIG. 12.

### Example 9

### Enhancement of Statin Effectiveness

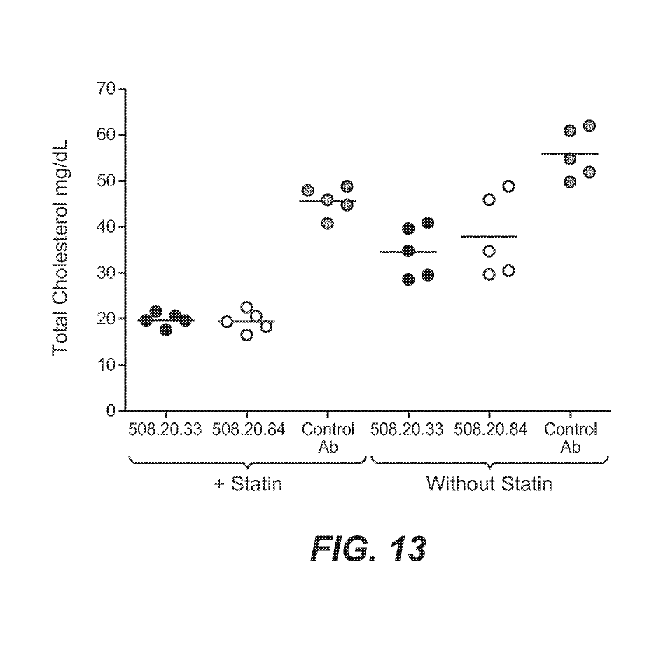

This experiment demonstrates that a combination of anti-PCSK9 antibody and statin results in a greater reduction in total cholesterol level compared to anti-PCSK9 antibody alone or statin alone treatments. See e.g., FIG. 13. Eight weeks old male C57BL/6J mice was purchased from Jackson Laboratory. The mice were grouped into 2 different groups. The non-statin mice received control diet, while statin groups received 0.2% of lovastatin in the diet for 2 weeks prior to antibody administration (Bioserve, Frenchtown, N.J.). All the mice were pre-bled and mice were randomized based on equal average cholesterol level. Mice were bled on day 3 and the total cholesterol levels were assayed using INFINITY™ Cholesterol Reagent (Fisher Diagnostics, Middletown, Va.).

The anti-PCSK9 antibodies showed significant cholesterol lowering effect. Statin alone treatment resulted in modest reduction in total cholesterol level, compared to non-statin groups. The combination of statin plus anti-PCSK9 antibody resulted in an additional reduction compared to anti-PCSK9 alone in total cholesterol level. See FIG. 13.

### Example 10

### X-Ray Crystal Structure of PCSK9 Bound to Fab Fragment of Anti-PCSK9 Antibody

Protein Purification and Crystallization

210 g of frozen cell paste from 10 L E. coli expression were thawed in 1 L of lysis buffer (PBS/25 mM EDTA/1 mM PMSF). Cells were disrupted by Tissuemizer (30 seconds) and the resulting slurry was passed through a microfluidizer twice. Insoluble matter was pelleted by centrifugation. Clarified lysate (250 mL at a time) was loaded onto a Protein G column (cat#17-0618-05, GE Healthcare) at 5 mL/min. The column was then washed with 100 mL of lysis buffer before eluting the bound Fab fragment of anti-PCSK9 antibody with 150 mL of elution buffer (0.58% acetic acid). 25 mL fractions were collected during elution. Fractions containing Fab fragment of anti-PCSK9 antibody were pooled after SDS PAGE analysis.

5 mL prepacked SPHP column (GE Healthcare, cat#17-1152-01) were equilibrated with 50 ml of Buffer A (20 mM MES pH5.5). Pooled fractions from the prior step were loaded onto the column at 3 mL/min. The column was washed with Buffer A to baseline. Bound Fab fragment was eluted with buffer B (20 mM MES pH 5.5, 1M NaCl) using a gradient from 0% to 100% buffer B in 20 column volumes. 2 mL fractions were collected during elution. The fractions containing the protein (determined using SDS-PAGE) were pooled and concentrated to 5 mL before loaded onto a 320 mL S75 gel filtration column that had been pre-equilibrated with sizing buffer (20 mM Hepes 7.2, 150 mM NaCl). The sizing buffer was run continuously at 1.5 mL/min for 220 mins while collecting 2 mL fractions. The peak fractions (A280) were analyzed using SDS-PAGE.

Human PCSK9 (Genbank EF692496) complementary deoxyribonucleic acids (cDNAs) containing a histidine (His)₈ C-terminal tag (SEQ ID NO:32) were inserted into a mammalian expression vector (pRKS) with a cytomegalovirus (CMV) promoter using standard molecular biology techniques. Protein was expressed by transient transfection of Chinese hamster ovary

(CHO) cells and purified from conditioned media using affinity chromatography on a nickel-nitrilotriacetic-agarose column (Qiagen) followed by gel filtration on a Sephacryl S-200 column (GE Healthcare). The correct masses of purified proteins were verified by sodium dodecyl sulfate polyacrylamide gel electrophoresis (SDS-PAGE) and the accuracy of amino acid sequences were confirmed by N-terminal sequencing.

The purified Fab fragment of anti-PCSK9 antibody and 6.9 mg of PCSK9 protein were mixed in 2-fold molar excess of the Fab fragment and incubated at 4° C. for 1 hour before concentration to 5 mL. The concentrated mixture was then loaded onto a Superdex 200 size exclusion column (cat#17-1071-01, GE Healthcare) pre-equilibrated with sizing buffer. The sizing buffer was continuously run at 1.5 mL/min for 220 mins while collecting 2 mL fractions. The peak fractions (A280) containing both PCSK9 and Fab fragment of anti-PCSK9 antibody (SDS-PAGE) were pooled and concentrated to 20 mg/mL. The concentrated complex was then used to set up crystallization trials. Initial crystals were formed from a 1:1 mixture between protein and reservoir containing 1.3 M potassium/sodium phosphate at pH 7 using sitting drops. Crysals were optimized by varying the protein:reservoir ratio in hanging drops. A selected crystal was treated with mother liquor supplumented with 25% glycerol and preserved in liquid nitrogen.

Structure Determination of the PCSK9:Fab Fragment of Anti-PCSK9 Antibody Complex

Diffraction data extending to about 3.5 Å resolution were collected at synchrotron beamline SSRL 7-1 and integrated and scaled in space group 1222. Approximate phases were obtained by the method of molecular replacement, using the previously reported structure of PCSK9 (Hampton et al., PNAS104:14609-9 (2007), pdb accession code 2QTW) and the previously reported structure of an antibody Fv fragment (Eigenbrot et al., J Mol Biol 229:969-95 (1993), pdb accession code 1FVC). The constant region of Fab fragment of anti-PCSK9 antibody was placed as a rigid body using a part of a previously reported homologous structure (Eigenbrot et al. supra, pdb accession code 1FVD) after partial refinement had improved phases. The final refined structure has crystallographic R-values of 25 & 30%. Data collection and refinement statistics appear in Table 1 below.

TABLE 1

| Data collection | |
|---|---|
| space group | I222 |
| unit cell (Å, °) | a = 92.283, b = 142.523, c = 253.983 |
| V_{M} (Å³/Dalton) | 2.8 |
| Resolution (Å) | 40-3.5 (3.63-3.50) |
| Rsym^{a,b} | 0.184 (0.807) |
| Number of observations | 157526 |
| Unique reflections | 21579 |
| Completeness (%)^{b} | 100 (100) |
| I/σI^{b} | 11 (2.6) |
| Wilson B (Å²) | 58 |
| Refinement | |
| Resolution (Å) | 40-3.5 |
| Number of reflections | 20644 |
| (F > 0σ(F)) | |
| Final R^{c}, R_{FREE} | 0.247, 0.295 |
| complexes/asymmetric unit | 1 |
| protein residues | 994 |
| solvent molecules | 0 |
| atoms | 7463 |
| Mean B-factor (Å²) | 86 |
| Rmsd bonds (Å) | 0.007 |
| Rmsd angles (°) | 1.1 |
| Rmsd bonded Bs (Å²) | 2.4/1.9 |
| Number of TLS groups | 4 |
| Ramachandran (%) | 81.5/16.8/0.6/1.1 |

^{a}Rsym = Σ||I| − |<I>||/Σ|<I>|, where I is the intensity of a single observation and <I> the average intensity for symmetry equivalent observations.
^{b}In parenthesis, for the highest resolution shell.
^{c}R = Σ|Fo − Fc|/Σ|Fo|, where Fo and Fc are observed and calculated structure factor amplitudes, respectively. R_{FREE} is calculated as R for reflections sequestered from refinement.

Determination of Epitope on PCSK9 from the X-Ray Structure

A 4A criterion was applied using the molecular analysis program PyMOL. PCSK9 residues within 4A of any part of the Fab fragment of anti-PCSK9 antibody were determined as an epitope. Based on the analysis, the epitope comprises one or more of the following residues: R194, E195, D238, A239, A341, Q342, E366, D367, I369, S376, T377, C378, F379, S381 and H391 of human PCSK9.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. An anti-PCSK9 antibody or an antibody fragment that binds to PCSK9 comprising a variable domain comprising at least one, two, three, four, five or six hypervariable region (HVR) sequences selected from the group consisting of:
(i) HVR-H1 comprising GFTFX₁X₂X₃X₄IH (SEQ ID NO:28), wherein X₁ is S or T; X₂ is G, R or S; X₃ is H, T or Y; X₄ is A or T;
(ii) HVR-H2 comprising RISPANGNTNYADSVKG (SEQ ID NO:4);
(iii) HVR-H3 comprising WIGSRELYIMDY (SEQ ID NO:5);
(iv) HVR-L1 comprising RASQDVSX₁AVA (SEQ ID NO:29), wherein X₁ is S or T;
(v) HVR-L2 comprising SASX₁LYS (SEQ ID NO:30), wherein X₁ is F or S; and
(vi) HVR-L3 comprising QQSYX₁X₂X₃X₄T (SEQ ID NO:31), wherein X₁ is P, R or T;X₂ is A, I, S or T; X₃ is L, P or Q; X₄ is A, H, P or S.

2. An anti-PCSK9 antibody or an antibody fragment that binds to PCSK9 comprising a variable domain comprising the following six HVR sequences:
(i) HVR-H1 comprising GFTFX₁X₂X₃X₄₁H (SEQ ID NO:28), wherein X₁ is S or T; X₂ is G, R or S; X₃ is H, T or Y; X₄ is A or T;
(ii) HVR-H2 comprising RISPANGNTNYADSVKG (SEQ ID NO:4);
(iii) HVR-H3 comprising WIGSRELYIMDY (SEQ ID NO:5);
(iv) HVR-L1 comprising RASQDVSX₁AVA (SEQ ID NO:29), wherein X₁ is S or T;
(v) HVR-L2 comprising SASX₁LYS (SEQ ID NO:30), wherein X₁ is F or S; and
(vi) HVR-L3 comprising QQSYX₁X₂X₃X₄T (SEQ ID NO:31), wherein X₁ is P, R or T; X₂ is A, I, S or T; X₃ is L, P or Q; X₄ is A, H, P or S.

3. The antibody of claim 1, wherein the antibody comprises (a) HVR-H1 comprising the amino acid sequence of SEQ ID NO:1, SEQ ID NO:2 or SEQ ID NO:3, (b) HVR-H2 comprising the amino acid sequence of SEQ ID NO:4, and (c) HVR-H3 comprising the amino acid sequence of SEQ ID NO:5.

4. The antibody of claim 3, further comprising (a) HVR-L1 comprising the amino acid sequence of SEQ ID NO:6 or SEQ ID NO:7; (b) HVR-L2 comprising the amino acid sequence of SEQ ID NO:8 or SEQ ID NO:26; and (c) HVR-L3 comprising the amino acid sequence of SEQ ID NO:9, SEQ ID NO:10, SEQ ID NO:11, SEQ ID NO:12, SEQ ID NO:13 or SEQ ID NO:14.

5. The antibody of claim 1, comprising (a) HVR-L1 comprising the amino acid sequence of SEQ ID NO:6 or SEQ ID NO:7; (b) HVR-L2 comprising the amino acid sequence of SEQ ID NO:8 or SEQ ID NO:26; and (c) HVR-L3 comprising the amino acid sequence of SEQ ID NO: 9, SEQ ID NO:10, SEQ ID NO:11, SEQ ID NO:12, SEQ ID NO:13 or SEQ ID NO:14.

6. The antibody of claim 5, further comprising (a) HVR-H1 comprising the amino acid sequence of SEQ ID NO:1, SEQ ID NO:2 or SEQ ID NO:3, (b) HVR-H2 comprising the amino acid sequence of SEQ ID NO:4, and (c) HVR-H3 comprising the amino acid sequence of SEQ ID NO:5.

7. The anti-PCSK9 antibody of claim 1, wherein the antibody comprises:
(1) an HVR-H1 comprising the amino acid sequence of SEQ ID NO:1;
(2) an HVR-H2 comprising the amino acid sequence of SEQ ID NO:4;
(3) an HVR-H3 comprising the amino acid sequence of SEQ ID NO:5;
(4) an HVR-L1 comprising the amino acid sequence of SEQ ID NO:7;
(5) an HVR-L2 comprising the amino acid sequence of SEQ ID NO:8; and
(6) an HVR-L3 comprising the amino acid sequence of SEQ ID NO:10.

8. The anti-PCSK9 antibody of claim 1, wherein the antibody comprises:
(1) an HVR-H1 comprising the amino acid sequence of SEQ ID NO:1;
(2) an HVR-H2 comprising the amino acid sequence of SEQ ID NO:4;
(3) an HVR-H3 comprising the amino acid sequence of SEQ ID NO:5;
(4) an HVR-L1 comprising the amino acid sequence of SEQ ID NO:7;
(5) an HVR-L2 comprising the amino acid sequence of SEQ ID NO:8; and
(6) an HVR-L3 comprising the amino acid sequence of SEQ ID NO:11.

9. The anti-PCSK9 antibody of claim 1, wherein the antibody comprises:
(1) an HVR-H1 comprising the amino acid sequence of SEQ ID NO:2;
(2) an HVR-H2 comprising the amino acid sequence of SEQ ID NO:4;
(3) an HVR-H3 comprising the amino acid sequence of SEQ ID NO:5;
(4) an HVR-L1 comprising the amino acid sequence of SEQ ID NO:7;
(5) an HVR-L2 comprising the amino acid sequence of SEQ ID NO:8; and
(6) an HVR-L3 comprising the amino acid sequence of SEQ ID NO:12.

10. The anti-PCSK9 antibody of claim 1, wherein the antibody comprises:
(1) an HVR-H1 comprising the amino acid sequence of SEQ ID NO:3;
(2) an HVR-H2 comprising the amino acid sequence of SEQ ID NO:4;
(3) an HVR-H3 comprising the amino acid sequence of SEQ ID NO:5;
(4) an HVR-L1 comprising the amino acid sequence of SEQ ID NO:7;
(5) an HVR-L2 comprising the amino acid sequence of SEQ ID NO:8; and
(6) an HVR-L3 comprising the amino acid sequence of SEQ ID NO:13.

11. The anti-PCSK9 antibody of claim 1, wherein the antibody comprises:
(1) an HVR-H1 comprising the amino acid sequence of SEQ ID NO:1;
(2) an HVR-H2 comprising the amino acid sequence of SEQ ID NO:4;
(3) an HVR-H3 comprising the amino acid sequence of SEQ ID NO:5;
(4) an HVR-L1 comprising the amino acid sequence of SEQ ID NO:7;
(5) an HVR-L2 comprising the amino acid sequence of SEQ ID NO:8; and
(6) an HVR-L3 comprising the amino acid sequence of SEQ ID NO:14.

12. The anti-PCSK9 antibody of claim 1, comprising (a) a VH sequence having at least 95% sequence identity to the amino acid sequence of SEQ ID NO:15, SEQ ID NO:16, SEQ ID NO:17, or SEQ ID NO:27; or (b) a VL sequence having at least 95% sequence identity to the amino acid sequence of SEQ ID NO: 18, SEQ ID NO:19, SEQ ID NO:20, SEQ ID NO:21, SEQ ID NO:22 or SEQ ID NO:23.

13. The anti-PCSK9 antibody of claim 12, comprising a VH sequence of SEQ ID NO:15, SEQ ID NO:16, SEQ ID NO:17 or SEQ ID NO:27.

14. The anti-PCSK9 antibody of claim 12, comprising a VL sequence of SEQ ID NO: 18, SEQ ID NO:19, SEQ ID NO:20, SEQ ID NO:21, SEQ ID NO:22 or SEQ ID NO:23.

15. The anti-PCSK9 antibody of claim 1 comprising a VH sequence of SEQ ID NO:15 and a VL sequence of SEQ ID NO:19.

16. The anti-PCSK9 antibody of claim 1 comprising a VH sequence of SEQ ID NO:27 and a VL sequence of SEQ ID NO:20.

17. The anti-PCSK9 antibody of claim 1 comprising a VH sequence of SEQ ID NO:16 and a VL sequence of SEQ ID NO:21.

18. The anti-PCSK9 antibody of claim 1 comprising a VH sequence of SEQ ID NO:17 and a VL sequence of SEQ ID NO:22.

19. The anti-PCSK9 antibody of claim 1 comprising a VH sequence of SEQ ID NO:27 and a VL sequence of SEQ ID NO:23.

20. The anti-PCSK9 antibody of claim 1, wherein the antibody is a monoclonal antibody.

21. The anti-PCSK9 antibody of claim 1, wherein the antibody is humanized.

22. The anti-PCSK9 antibody of claim 1, wherein the antibody is human.

23. The anti-PCSK9 antibody of claim 1, wherein the antibody is an antibody fragment selected from a Fab, Fab′-SH, Fv, scFv or (Fab′)₂ fragment.

24. The anti-PCSK9 antibody of claim 1, wherein at least a portion of the framework sequence is a human consensus framework sequence.

25. An isolated nucleic acid encoding the anti-PCSK9 antibody of claim 1.

26. A vector comprising the nucleic acid of claim 25.

27. The vector of claim 26, wherein the vector is an expression vector.

28. A host cell comprising the vector of claim 26.

29. The host cell of claim 28, wherein the host cell is prokaryotic.

30. The host cell of claim 28, wherein the host cell is eukaryotic.

31. A method for making an anti-PCSK9 antibody, said method comprising culturing a host cell comprising a vector comprising a nucleic acid encoding the anti-PCSK9 antibody of claim 1 under conditions suitable for expression of the nucleic acid encoding the anti-PCSK9 antibody.

32. The method of claim 31, further comprising recovering the anti-PCSK9 antibody from the host cell.

33. A pharmaceutical composition comprising the anti-PCSK9 antibody of claim 1 and a pharmaceutically acceptable carrier.

34. A method of reducing LDL-cholesterol level in a subject, said method comprising administering to the subject an effective amount of the anti-PCSK9 antibody of claim 1.

35. A method of treating cholesterol related disorder in a subject, said method comprising administering to the subject an effective amount of the anti-PCSK9 antibody of claim 1.

36. A method of treating hypercholesterolemia in a subject, said method comprising administering to the subject an effective amount of the anti-PCSK9 antibody of claim 1.

37. The method of claim 34, further comprising administering to the subject an effective amount of a second medicament, wherein the anti-PCSK9 antibody is the first medicament.

38. The method of claim 37, wherein the second medicament elevates the level of LDLR.

39. The method of claim 37, wherein the second medicament reduces the level of LDL-cholesterol.

40. The method of claim 37, wherein the second medicament comprises a statin.

41. The method of claim 40, wherein the statin is selected from the group consisting of atorvastatin, fluvastatin, lovastatin, mevastatin, pitavastatin, pravastatin, rosuvastatin, simvastatin, and any combination thereof.

42. The method of claim 37, wherein the second medicament elevates the level of HDL-cholesterol.

43. A method of inhibiting binding of PCSK9 to LDLR in a subject, said method comprising administering to the subject an effective amount of the anti-PCSK9 antibody of claim 1.

44. A method of detecting PCSK9 protein in a sample, said method comprising
(a) contacting the sample with the antibody of claim 1; and
(b) detecting formation of a complex between the anti-PCSK9 antibody and the PCSK9 protein.

45. An anti-PCSK9 antibody or an antibody fragment that binds to PCSK9, wherein the antibody or antibody fragment binds to an epitope of PCSK9 that comprises:
(i) at least one residue selected from the group consisting of R194 and E195 of human PCSK9,
(ii) at least one residue selected from the group consisting of D238 and A239 of human PCSK9,
(iii) at least one residue selected from the group consisting of A341 and Q342 of human PCSK9, and
(iv) at least one residue selected from the group consisting of E366, D367, I369, S376, T377, C378, F379, S381 and H391 of human PCSK9.

46. The antibody of claim 45, wherein the epitope of PCSK9 comprises one or more of E366, D367 or H391.

47. The antibody of claim 46, wherein epitope of PCSK9 comprises E366, D367 and H391.


SEQUENCE LISTING (32 sequences)

SEQ ID NO: 1 - protein, 10 aa, Artificial Sequence
  Description of Artificial Sequence Synthetic peptide
GFTFTGYAIH

SEQ ID NO: 2 - protein, 10 aa, Artificial Sequence
  Description of Artificial Sequence Synthetic peptide
GFTFTRHTIH

SEQ ID NO: 3 - protein, 10 aa, Artificial Sequence
  Description of Artificial Sequence Synthetic peptide
GFTFSSTAIH

SEQ ID NO: 4 - protein, 17 aa, Artificial Sequence
  Description of Artificial Sequence Synthetic peptide
RISPANGNTNYADSVKG

SEQ ID NO: 5 - protein, 12 aa, Artificial Sequence
  Description of Artificial Sequence Synthetic peptide
WIGSRELYIMDY

SEQ ID NO: 6 - protein, 11 aa, Artificial Sequence
  Description of Artificial Sequence Synthetic peptide
RASQDVSSAVA

SEQ ID NO: 7 - protein, 11 aa, Artificial Sequence
  Description of Artificial Sequence Synthetic peptide
RASQDVSTAVA

SEQ ID NO: 8 - protein, 7 aa, Artificial Sequence
  Description of Artificial Sequence Synthetic peptide
SASFLYS

SEQ ID NO: 9 - protein, 9 aa, Artificial Sequence
  Description of Artificial Sequence Synthetic peptide
QQSYTTPPT

SEQ ID NO: 10 - protein, 9 aa, Artificial Sequence
  Description of Artificial Sequence Synthetic peptide
QQSYPAPAT

SEQ ID NO: 11 - protein, 9 aa, Artificial Sequence
  Description of Artificial Sequence Synthetic peptide
QQSYPSPAT

SEQ ID NO: 12 - protein, 9 aa, Artificial Sequence
  Description of Artificial Sequence Synthetic peptide
QQSYRIQPT

SEQ ID NO: 13 - protein, 9 aa, Artificial Sequence
  Description of Artificial Sequence Synthetic peptide
QQSYPALHT

SEQ ID NO: 14 - protein, 9 aa, Artificial Sequence
  Description of Artificial Sequence Synthetic peptide
QQSYPAPST

SEQ ID NO: 15 - protein, 121 aa, Artificial Sequence
  Description of Artificial Sequence Synthetic polypeptide
EVQLVESGGGLVQPGGSLRLSCAASGFTFTGYAIHWVRQAPGKGLEWVGRISPANGNTNYADSVKGRFTISADTSKNTAYLQMNSLRAEDTAVYYCARWIGSRELYIMDYWGQGTLVTVSS

SEQ ID NO: 16 - protein, 121 aa, Artificial Sequence
  Description of Artificial Sequence Synthetic polypeptide
EVQLVESGGGLVQPGGSLRLSCAASGFTFTRHTIHWVRQAPGKGLEWVARISPANGNTNYADSVKGRFTISADTSKNTAYLQMNSLRAEDTAVYYCARWIGSRELYIMDYWGQGTLVTVSS

SEQ ID NO: 17 - protein, 121 aa, Artificial Sequence
  Description of Artificial Sequence Synthetic polypeptide
EVQLVESGGGLVQPGGSLRLSCAASGFTFSSTAIHWVRQAPGKGLEWVARISPANGNTNYADSVKGRFTISADTSKNTAYLQMNSLRAEDTAVYYCARWIGSRELYIMDYWGQGTLVTVSS

SEQ ID NO: 18 - protein, 108 aa, Artificial Sequence
  Description of Artificial Sequence Synthetic polypeptide
DIQMTQSPSSLSASVGDRVTITCRASQDVSSAVAWYQQKPGKAPKLLIYSASSLYSGVPSRFSGSRSGTDFTLTISSLQPEDFATYYCQQSYTTPPTFGQGTKVEIKR

SEQ ID NO: 19 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYPAPATFGQGTKVEIKR

SEQ ID NO: 20 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYPSPATFGQGTKVEIKR

SEQ ID NO: 21 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYRIQPTFGQGTKVEIKR

SEQ ID NO: 22 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYPALHTFGQGTKVEIKR

SEQ ID NO: 23 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYPAPSTFGQGTKVEIKR

SEQ ID NO: 24 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 25 - DNA, 3636 bp, Homo sapiens
cagcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgcgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcct

SEQ ID NO: 26 - protein, 7 aa, Artificial Sequence
  Description of Artificial Sequence Synthetic peptide
SASSLYS

SEQ ID NO: 27 - protein, 121 aa, Artificial Sequence
  Description of Artificial Sequence Synthetic polypeptide
EVQLVESGGGLVQPGGSLRLSCAASGFTFTGYAIHWVRQAPGKGLEWVARISPANGNTNYADSVKGRFTISADTSKNTAYLQMNSLRAEDTAVYYCARWIGSRELYIMDYWGQGTLVTVSS

SEQ ID NO: 28 - protein, 10 aa, Artificial Sequence
  Description of Artificial Sequence Synthetic peptide
GFTFXXXXIH

SEQ ID NO: 29 - protein, 11 aa, Artificial Sequence
  Description of Artificial Sequence Synthetic peptide
RASQDVSXAVA

SEQ ID NO: 30 - protein, 7 aa, Artificial Sequence
  Description of Artificial Sequence Synthetic peptide
SASXLYS

SEQ ID NO: 31 - protein, 9 aa, Artificial Sequence
  Description of Artificial Sequence Synthetic peptide
QQSYXXXXT

SEQ ID NO: 32 - protein, 8 aa, Artificial Sequence
  Description of Artificial Sequence Synthetic 8xHis tag
HHHHHHHH